(19) United States
(12) Patent Application Publication
(10) Pub. No.: US 2022/0068438 A1
(43) Pub. Date: Mar. 3, 2022

(54) DEEP LEARNING AND ALIGNMENT OF SPATIALLY-RESOLVED WHOLE TRANSCRIPTOMES OF SINGLE CELLS

(71) Applicants: The Broad Institute, Inc., Cambridge, MA (US); Massachusetts Institute of Technology, Cambridge, MA (US)

(72) Inventors: Aviv Regev, Cambridge, MA (US); Tommaso Biancalani, Cambridge, MA (US); Gabriele Scalia, Cambridge, MA (US); Lorenzo Buffoni, Cambridge, MA (US)

(21) Appl. No.: 17/459,368

(22) Filed: Aug. 27, 2021

Related U.S. Application Data
(60) Provisional application No. 63/070,993, filed on Aug. 27, 2020.

(51) Int. Cl. G16B 40/30; G16B 45/00; G06N 3/08

(52) U.S. Cl. CPC G16B 40/30; G06N 3/088; G16B 45/00

(57) ABSTRACT

The present invention provides for methods, systems and computer products for aligning single cell data with spatial data to generate spatial maps of cell types and gene expression at single cell resolution. The invention further provides for mapping to common coordinate frameworks.

### CROSS-REFERENCE TO RELATED APPLICATIONS

This application claims the benefit of U.S. Provisional Application No. 63/070,993, filed Aug. 27, 2020. The entire contents of the above-identified application are hereby fully incorporated herein by reference.

### STATEMENT REGARDING FEDERALLY SPONSORED RESEARCH

This invention was made with government support under Grant Nos. MH114821 and OD026673 awarded by the National Institutes of Health. The government has certain rights in the invention.

### TECHNICAL FIELD

The subject matter disclosed herein is generally directed to aligning single cell data with spatial data to generate a map having single cell resolution and relating those maps to histological and anatomical positions in the context of an organ's common coordinate framework.

### BACKGROUND

An Human Cell Atlas, and especially a Brain Cell Atlas, should combine high resolution molecular and histological mapping with anatomical and functional data. Achieving this goal relies critically on our ability to relate different levels of biological organization and data modalities to each other. In this context, molecular data, for example mRNA levels, has the potential to provide a unifying connector, especially if it can be quantified both spatially and at single cell resolution. In addition, such spatial measurements then relate cellular features across scales, including histological and anatomical, by mapping through a Common Coordinate Framework (CFF) of an organ⁹. However, to achieve this ultimate goal, two challenges need to be overcome: obtain molecular profiles at high spatial resolution, and relate those to both histological and anatomical information.

Advances in single cell and spatial genomics¹⁰ opened the way to high resolution spatial profiles, but each of the currently available technologies only addresses some of the challenge of resolving whole transcriptomes in space at single-cell resolution. On the one hand, single cell and single nucleus RNA-seq (sc/snRNA-seq) profile single cells transcriptome-wide, from which we can recover cell types¹¹, gene programs^{12,13}, and developmental relations^{14,15}, but by necessity loses spatial information. Conversely, spatial technologies resolve transcriptomes in space, but are limited in either gene throughput or spatial resolution. In general, targeted in situ technologies (e.g., ISS¹⁶, MERFISH¹, smFISH³, osmFISH¹⁷, STARMap², Targeted ExSeq¹⁸, seqFISH+¹⁹) are limited in the number measured genes measured, which must be pre-selected and are typically in the hundreds to thousands range, whereas adding more probes can reduce accuracy for some genes². Spatial Transcriptomics methods (e.g., Spatial Transcriptomics (ST)⁴ (now available commercially as Visium), Slide-seq²⁰, or High Definition Spatial Trascriptomics²¹) spatially barcode entire transcriptomes, but with limited capture rate (and substantial “dropouts”, which increase at higher resolution) and a spatial resolution larger than a single cell, ranging from 50-100 μm for ST to 10 μm for Slide-seq.

Computational methods have previously bridged this gap by combining single cell and spatial measurements²²⁻²⁵. With targeted, high resolution, spatial measurements, mapping provides spatial measurements for additional genes based on their (mapped) single cell profiles. These methods can reconstruct key landmark genes by leveraging local alignment in transcriptome space²²⁻²⁴, or hypotheses such as continuity in gene expression²⁵. However, intrinsically sparse or granularly distributed genes are difficult to predict. For measurements at coarse spatial resolution, computational methods aim to deconvolve these data^{20,26}, by either learning a program dictionary²⁰ or a probability distribution of the data²⁶, to infer a cell type composition within a spatial voxel. However, deconvolution is hindered by spatial “dropouts”, where cells types defined by sparse or dim markers are not correctly detected²⁷.

In many cases, only histological data is directly available for the specimens collected as part of single cell atlases, but those can serve as a bridge to pre-existing atlases, with measured in situ hybridization (ISH) data, and rich anatomical annotations in the context of a Common Coordinate Framework, as in the case of the Allen Brain Atlas²⁸. Using these data should allow relating cellular features (e.g., gene expression, cell types) to the histological or organ scale, especially in the brain. However, typical methods from computer vision for registration of medical images^{29,30} require human supervision, such as identification of a few corresponding anatomical landmarks in experimental and atlas images. Such supervision, albeit minimal, prevents complete automation. A common strategy to remove supervision uses machine learning for identifying the few key landmarks required in registration, as has been shown for registration of sagittal mouse brain slides³¹. However, this method is not suitable for images that are torn or contain holes, for example, if tissue has been first dissected for profiling sc/snRNA-seq data.

Citation or identification of any document in this application is not an admission that such a document is available as prior art to the present invention.

### SUMMARY

In one aspect, the present invention provides for a method of aligning single cell data with spatial data to generate spatial maps of cell types and gene expression at single cell resolution comprising: by one or more computing devices: receiving single cell data obtained from a specimen from a specific anatomical region or tissue type; receiving spatial data obtained from the same specimen or the same anatomical region or tissue type from a different specimen, wherein the single cell data and spatial data comprises at least one shared measured features; aligning the single cell data to the spatial data based on one or more of the shared features using unsupervised deep learning nonlinear optimization; and generating a spatial map wherein the single cell data constitutes the new spatial data. In certain embodiments, the unsupervised deep learning non-linear optimization uses one or more similarity functions. In certain embodiments, the one or more similarity functions comprise Kullback-Leibler (KL) divergence and cosine similarity. In certain embodiments, the method further comprises validating the spatial map by predicting the expression of one or more holdout genes. In certain embodiments, the method further comprises applying a first learned spatial map to a second spatial map to increase the resolution of the second map. In certain embodiments, the method further comprises relating the spatial map to histological and anatomical data for the same specimen or the same anatomical region or tissue type from a different specimen. In certain embodiments, the method further comprises registering the spatial maps on an anatomically annotated common coordinate framework. In certain embodiments, registering comprises using a Siamese neural network and a semantic segmentation algorithm.

In certain embodiments, the shared features comprise gene expression, accessible chromatin, an epigenetic mark, and/or a combination thereof. In certain embodiments, the single cell data and spatial data comprises at least 10 shared features. In certain embodiments, the single cell data comprises the expression of greater than 10,000 genes and the spatial data comprises spatial expression of less than 100 genes.

In certain embodiments, the single cell data comprises single cell RNA-seq data (scRNA-seq) or single nucleus RNA-seq data (snRNA-seq). In certain embodiments, the single cell data comprises single cell ChIP. In certain embodiments, the single cell data comprises single cell ATAC-seq. In certain embodiments, the single cell data comprises single cell proteomics. In certain embodiments, the spatial data is coarse grained. In certain embodiments, the spatial data comprises in situ Hybridization (ISH), Single-molecule Fluorescence in situ Hybridization (smFISH), Spatial Transcriptomics (Visium), STARmap and/or MERFISH. In certain embodiments, the single cell data is multi-modal single cell data. In certain embodiments, the multi-modal data is single cell RNA-seq and chromatin accessibility data (SHARE-seq). In certain embodiments, the multi-modal data is single cell RNA-seq and proteomics data (CITE-seq). In certain embodiments, the multi-modal data is single cell RNA-seq and patch-clamping electrophysiological recording and morphological analysis of single neurons (Patch-seq).

In certain embodiments, the method comprises receiving an input of one or more types of genes. In certain embodiments, the method comprises generating a single cell matrix and a mapping matrix. In certain embodiments, the method comprises: determining a quantity of cells in the single cell data; determining a quantity of cells in the spatial data; and applying a mapping filter if the quantity of cells in the single cell data is greater than the quantity of cells in the spatial data. In certain embodiments, applying the mapping filter comprises: determining a probability of finding each cell of the single cell data in a voxel of the spatial data; assigning a filter value to each cell of the single cell data based on the determined probability; generating a filter vector; applying a sigmoid function to the filter vector; and filtering the single cell matrix and the mapping matrix.

In another aspect, the present invention provides for a system to align single cell data with spatial data to generate spatial maps of cell types at single cell resolution comprising: a storage device; and a processor communicatively coupled to the storage device, wherein the processor executes application code instructions that are stored in the storage device to cause the system to: receive single cell data obtained from a specimen from a specific anatomical region or tissue type; receive spatial data obtained from the same specimen or the same anatomical region or tissue type from a different specimen, wherein the single cell data and spatial data comprises at least one shared feature; align the single cell data to the spatial data based on one or more of the shared features using unsupervised deep learning non linear optimization; and generate a spatial map wherein the single cell data constitutes the new spatial data. In certain embodiments, the unsupervised deep learning non-linear optimization uses one or more similarity functions. In certain embodiments, the one or more similarity functions comprise Kullback-Leibler (KL) divergence and cosine similarity. In certain embodiments, the system further comprises application code instructions to validate the spatial map by predicting the expression of one or more holdout genes. In certain embodiments, the system further comprises application code instructions to apply a first learned spatial map to a second spatial map to increase the resolution of the second map. In certain embodiments, the system further comprises application code instructions to relate the spatial map to histological and anatomical data for the same specimen or the same anatomical region or tissue type from a different specimen. In certain embodiments, the system further comprises application code instructions to register the spatial maps on an anatomically annotated common coordinate framework. In certain embodiments, registering comprises application code instructions to use a Siamese neural network and a semantic segmentation algorithm.

In certain embodiments, the shared features comprise gene expression, accessible chromatin, an epigenetic mark, and/or a combination thereof. In certain embodiments, the single cell data and spatial data comprises at least 10 shared features. In certain embodiments, the single cell data comprises the expression of greater than 10,000 genes and the spatial data comprises spatial expression of less than 100 genes. In certain embodiments, the single cell data comprises single cell RNA-seq data (scRNA-seq) or single nucleus RNA-seq data (snRNA-seq). In certain embodiments, the single cell data comprises single cell ChIP. In certain embodiments, the single cell data comprises single cell ATAC-seq. In certain embodiments, the single cell data comprises single cell proteomics. In certain embodiments, the spatial data is coarse grained. In certain embodiments, the spatial data comprises in situ Hybridization (ISH), Single-molecule Fluorescence in situ Hybridization (smFISH), Spatial Transcriptomics (Visium), STARmap and/or MERFISH. In certain embodiments, the single cell data is multi-modal single cell data. In certain embodiments, the multi-modal data is single cell RNA-seq and chromatin accessibility data (SHARE-seq). In certain embodiments, the multi-modal data is CITE-seq data. In certain embodiments, the multi-modal data is single cell RNA-seq and patch-clamping electrophysiological recording and morphological analysis of single neurons (Patch-seq).

In certain embodiments, the system comprises application code instructions to receive an input of one or more types of genes. In certain embodiments, the system comprises application code instructions to generate a single cell matrix and a mapping matrix. In certain embodiments, the system comprises application code instructions to: determine a quantity of cells in the single cell data; determine a quantity of cells in the spatial data; and apply a mapping filter if the quantity of cells in the single cell data is greater than the quantity of cells in the spatial data. In certain embodiments, applying the mapping filter comprises application code instructions to: determine a probability of finding each cell of the single cell data in a voxel of the spatial data; assign a filter value to each cell of the single cell data based on the determined probability; generate a filter vector; apply a sigmoid function to the filter vector; and filter the single cell matrix and the mapping matrix.

In another aspect, the present invention provides for a computer program product to align single cell data with spatial data to generate spatial maps of cell types at single cell resolution, comprising: a non-transitory computer-readable medium having computer-readable program instructions embodied thereon that, when executed by a computer, cause the computer to: receive single cell data obtained from a specimen from a specific anatomical region or tissue type; receive spatial data obtained from the same specimen or the same anatomical region or tissue type from a different specimen, wherein the single cell data and spatial data comprises at least one shared feature; align the single cell data to the spatial data based on one or more of the shared features using unsupervised deep learning non linear optimization; and generate a spatial map wherein the single cell data constitutes the new spatial data. In certain embodiments, the unsupervised deep learning non-linear optimization uses one or more similarity functions. In certain embodiments, the one or more similarity functions comprise Kullback-Leibler (KL) divergence and cosine similarity. In certain embodiments, the computer program product further comprises computer-readable program instructions to validate the spatial map by predicting the expression of one or more holdout genes. In certain embodiments, the computer program product further comprises computer-readable program instructions to apply a first learned spatial map to a second spatial map to increase the resolution of the second map. In certain embodiments, the computer program product further comprises computer-readable program instructions to relate the spatial map to histological and anatomical data for the same specimen or the same anatomical region or tissue type from a different specimen. In certain embodiments, the computer program product further comprises computer-readable program instructions to register the spatial maps on an anatomically annotated common coordinate framework. In certain embodiments, registering comprises computer-readable program instructions to use a Siamese neural network and a semantic segmentation algorithm.

In certain embodiments, the shared features comprise gene expression, accessible chromatin, an epigenetic mark, and/or a combination thereof. In certain embodiments, the single cell data and spatial data comprises at least 10 shared features. In certain embodiments, the single cell data comprises the expression of greater than 10,000 genes and the spatial data comprises spatial expression of less than 100 genes. In certain embodiments, the single cell data comprises single cell RNA-seq data (scRNA-seq) or single nucleus RNA-seq data (snRNA-seq). In certain embodiments, the single cell data comprises single cell ChIP. In certain embodiments, the single cell data comprises single cell ATAC-seq. In certain embodiments, the single cell data comprises single cell proteomics. In certain embodiments, the spatial data is coarse grained. In certain embodiments, the spatial data comprises in situ Hybridization (ISH), Single-molecule Fluorescence in situ Hybridization (smFISH), Spatial Transcriptomics (Visium), STARmap and/or MERFISH. In certain embodiments, the single cell data is multi-modal single cell data. In certain embodiments, the multi-modal data is single cell RNA-seq and chromatin accessibility data (SHARE-seq). In certain embodiments, the multi-modal data is CITE-seq data. In certain embodiments, the multi-modal data is single cell RNA-seq and patch-clamping electrophysiological recording and morphological analysis of single neurons (Patch-seq).

In certain embodiments, the computer program product comprises computer-readable program instructions to receive an input of one or more types of genes. In certain embodiments, the computer program product comprises computer-readable program instructions to generate a single cell matrix and a mapping matrix. In certain embodiments, the computer program product comprises computer-readable program instructions to: determine a quantity of cells in the single cell data; determine a quantity of cells in the spatial data; and apply a mapping filter if the quantity of cells in the single cell data is greater than the quantity of cells in the spatial data. In certain embodiments, applying the mapping filter comprises computer-readable program instructions to: determine a probability of finding each cell of the single cell data in a voxel of the spatial data; assign a filter value to each cell of the single cell data based on the determined probability; generate a filter vector; apply a sigmoid function to the filter vector; and filter the single cell matrix and the mapping matrix.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 is a block diagram depicting a portion of a communications and processing architecture of a typical system to learn and align spatially-resolved whole transcriptomes of single cells, in accordance with certain examples of the technology disclosed herein.

FIG. 2 is a block flow diagram depicting methods to align single cell data with spatial data to generate spatial maps of cell types at single cell resolution, in accordance with certain examples of the technology disclosed herein.

FIG. 3 is a block flow diagram depicting methods to generate spatial maps of cell types at single cell resolution, in accordance with certain examples of the technology disclosed herein.

FIG. 4 is a block flow diagram depicting methods to apply a mapping filter, in accordance with certain examples of the technology disclosed herein.

FIG. 5 is a block diagram depicting a computing machine and modules, in accordance with certain examples of the technology disclosed herein.

FIG. 6A-6F—Tangram learns spatial transcriptome-wide patterns at single cell resolution from sc/snRNA-seq data and corresponding spatial data. FIG. 6a. Overview. sc/snRNA-seq data and spatial data, collected from the same tissue are spatially aligned by comparing gene expression of their shared genes. FIG. 6b-f. Tangram use cases. b. Generating genome wide spatial patterns from gene signature data. Predicted expression patterns (color bar, normalized mRNA counts, Methods) for each of three genes not included in an input smFISH dataset validated against their corresponding images from the Allen ISH atlas (bottom). c. Correction of low-quality data for spatially measured genes. Predicted (top) and measured (bottom, by Visium) expression patterns (color bar, normalized mRNA counts, Methods) of four known markers whose correct localization is missing in direct Visium measurements, but recovered in the predicted patterns. d. Cell type localization. Spatial distribution of cell types defined by snRNA-seq (legend) mapped on a an smFISH brain slide. e. Single cell deconvolution of lower-resolution Spatial Transcriptomics. Predicted single cells (colored dots, legend) in each Visium voxel (grey circle) based on snRNA-seq data mapped onto a Visium slide. f. Spatially resolved chromatin patterns. Predicted spatial gene expression (top, color bar, normalized mRNA counts, Methods) and chromatin accessibility (bottom, color bar, normalized ATAC peak counts, Methods) by mapping the RNA component of SHARE-Seq data to a MERFISH slide.

FIG. 7A-7F—Tangram maps cells with high resolution MERFISH measurements and expands them to genome scale. FIG. 7a. Probabilistic mapping of snRNA-seq data on MERFISH data. Probability of mapping (color bar) of each cell subset (grey label) in each of 3 major categories. Bottom right: schematic of key layers. FIG. 7b. Deterministic mapping. MERFISH slide with segmented cells (dot) colored by the cell type annotation of the most likely snRNA-seq profile mapped on that position by tangram (legend). FIG. 7c,d. Predicted expression of test genes. c. Measured (top) and Tangram-predicted (bottom) expression (color bar signifies fluorescence at top and normalized mRNA counts at bottom, Methods) of select test gene (grey labels) with different extents of spatial correlation (bottom arrow, %) between measured and predicted patterns. d. Cumulative distribution function (CDF) of spatial correlation (x axis) between predicted and measured patterns for test genes. Dashed line: 75% of test genes are predicted with spatial correlation >40%. FIG. 7e. Predicted expression of test genes. Tangram-predicted (bottom) expression (top; color bar, normalized mRNA counts, Methods) and corresponding ISH images from the Allen Brain Atlas (bottom) for 11 genes not measured by MERFISH. FIG. 7f. Correction of low-quality spatial measurements. MERIFSH measured (top), Tangram-predicted (middle) and Allen Brain Atlas ISH, for genes whose predicted patterns differ from MERFISH measurement but match direct inspection of Allen ISH images (color bar, normalized mRNA counts, Methods).

FIG. 8A-8E—Correction of low-quality genes by mapping snRNA-seq on STARmap data. FIG. 8a. Probabilistic mapping of snRNA-seq data on STARmap data. Probability of mapping (color bar) of each cell subset (grey label) in each of 3 major categories. FIG. 8b. Deterministic mapping. STARmap slide with segmented cells (dot) colored by the cell type annotation of the most likely snRNA-seq profile mapped on that position by Tangram (legend). FIG. 8c. Measured (top) and Tangram-predicted (bottom) expression (color bar signifies fluorescence at top and normalized mRNA counts at bottom, Methods) of select test gene (grey labels). FIG. 8d. Correction of low-quality spatial measurements. Tangram-predicted test genes (left), STARmap measurements (middle) and Allen atlas images (right) (color bar, normalized mRNA counts, Methods) of four genes (grey labels) whose predicted patterns differ from STARmap measurement but match direct measurement by MERFISH. FIG. 8e. Predicted expression of test genes. Tangram-predicted (top) expression (top; color bar, normalized mRNA counts, Methods) and corresponding ISH images from the Allen Brain Atlas (bottom) for 6 genes not measured by STARmap.

FIG. 9A-9H—Mapping snRNA-seq data to Spatial Transcriptomics data (Visium) demonstrates deconvolution and imputation of dropouts. FIG. 9a. Single cell deconvolution. Predicted single cells (colored dots, legend) in each Visium voxel (grey circle) based on snRNA-seq data mapped onto Visium slide. Cell assignment within a voxel is random with respect to the specific segmented cell. FIG. 9b. Probabilistic mapping of snRNA-seq data on the Visium ROI. Probability of mapping (color bar) of each cell subset (grey label) in each of 3 major categories. FIG. 9c,d. Predicted expression of test and training genes. c. Normalized (i.e. unit area) distribution of single-gene spatial correlation coefficients (y axis) between Tangram-predicted and Visium measured patterns in training (orange) and test (blue) genes. d. Reducing the number of training genes decreases prediction performance. Spatial correlation (y axis, top) for training gene (orange) and test genes (blue), and scaled spatial correlation (y axis, bottom) for test genes (scaled by the correlation averaged across training genes) for Tangram models learned with different fractions of 1,237 input training genes (x axis). FIG. 9e-h. Impact of Visium data sparsity on prediction and correction. e. Tangram-predicted (top) and Visium measured (bottom) expression (color bar, normalized mRNA counts, Methods) of six select test genes (grey labels) with different extents of spatial correlation between measured and predicted patterns (top arrow, %) and of Visium data sparsity (bottom arrow, %). f. Spatial correlation of test genes is negatively correlated to sparsity in Visium data. Spatial correlation (y axis) between measured and predicted patterns for test genes (blue dots) and their corresponding measurement sparsity (x axis). Lines delineate three regions according to model performance. g. Few low sparsity genes are not predicted well. Tangram-predicted (top) and Visium measured (bottom) expression (color bar, normalized mRNA counts, Methods) of four genes (grey labels) with low sparsity that are not well-predicted by model (from region (ii) of panel f). h. Correction of low-quality spatial measurements. Tangram-predicted (left), Visium (middle) and MERFISH (right) measurements (color bar signifies fluorescence for MERFISH figure, normalized mRNA counts for all others, Methods), of two genes (grey labels) whose predicted patterns differ from Visium measurements but match direct measurement by MERFISH, and are highly-sparse in Visium measurements (from region (iii) of panel f).

FIG. 10A-10D—Tangram mapping of multi-omic SHARE-Seq profiles yields spatial patterns of chromatin accessibility and TF activity. FIG. 10a. Probabilistic mapping of SHARE-seq profiles on MERFISH data. Probability of mapping (color bar) of each cell subset (grey label) in each of 3 major categories based on the RNA component of SHARE-Seq profiles. FIG. 10b. Deterministic mapping. MERFISH slide with segmented cells (dot) colored by the cell type annotation of the most likely SHARE-Seq (RNA) profile mapped on that position by Tangram (legend). FIG. 10c. Predicted chromatin accessibility patterns. MERFISH measured expression (top, color bar, normalized fluorescence, Methods) and Tangram-predicted chromatin accessibility (bottom, color bar, normalized reads-in-peak count, Methods) of select genes (grey labels). FIG. 10d. Predicted TF activity patterns. Tangram predicted expression (top, color bar, mRNA counts) and activity normalized z-scores patterns (as inferred from snATAC-Seq, see Methods) (bottom, color bar, dimensionless) of select genes (grey labels) measured only by SHARE-Seq.

FIG. 11A-11D—A Siamese network model learns a similarity metric for brain sections based on anatomical landmarks in mouse brain images. FIG. 11a. Schematic of neural network architecture. A pair of images is fed to two convolutional encoders, which encode them into a 512-dimensional latent space. The image pair is labeled by the spatial coordinate (i.e., coronal depth) difference between the two images. FIG. 11b. The learned latent space is a 1D-manifold ordered by spatial coordinates. UMAP plot of the encoded training images from individual atlases (legend) colored by spatial depth (color bar). Insets illustrate four anatomically similar images from three different atlases and a test image. FIG. 11c. Predicted spatial coordinate distance (y axis) between a test image (inset, left panel) and each image of the training set obtained at different spatial coordinates (x axis). Dashed orange line: |ax+b| fit via mean square error minimization (a˜−0.96, b˜43) .The minimum of the fit is the predicted spatial coordinate (associated image is in the inset, right panel). FIG. 11d. Examples of model predictions (right) on test images (left) from the Macosko lab (first column; Methods), BrainMaps atlas (second column) and Allen ISH dataset (third and fourth columns).

FIG. 12A-12D—Anatomical region calling via semantic segmentation. FIG. 12a. Neural network model used for semantic segmentation. A U-net model is trained on mouse brain images from Allen atlas (left) to recognize five different classes on a mouse brain image (color legend, right). FIG. 12b. Augmentation pipeline. Each image undergoes a series of stochastic transformations including affine displacements, dropouts and color shifts (Methods). Four training samples are shown. FIG. 12c. Schematic of registration strategy. A segmentation mask of an experimental image is produced (I), the mask of each atlas image is extracted in parallel (II), the two masks are registered to each other (III); and finally the learned transformation is used to register the original images (IV). FIG. 12d. Prediction examples. Test images (left) and their predicted anatomical region calls (right).

FIG. 13A-13D—Tangram mapping of snRNA-seq profiles to histological and anatomical mouse brain atlases. FIG. 13a. Regions of interests. Nissl-stained images of coronal mouse brain slices highlighting the three regions of interest from which snRNA-seq data from the motor area were collected. FIG. 13b,c. Registration pipeline generates anatomical region and cell density maps. Anatomical region (b, color legend, from the Allen Common Coordinate Framework) and cell map (c, color bar, from the Blue Brain Cell Atlas) maps of each of the three dissected ROIs. FIG. 13d. Probabilistic mapping of snRNA-seq data on the ROI. Probability of mapping (color bar) of each cell subset (grey label) from each of 3 major categories within each ROI (rows).

FIG. 14A-14C—Mapping results on Visium data are consistent across three datasets. FIG. 14a. Consistent probabilistic maps across models trained from replicate datasets. Probability of mapping (color bar) of each cell subset (grey label) from each of 3 major categories in models trained separately from three Visium sections (rows). Section I is the same shown in FIG. 3b. FIG. 14b,c. Consistent deconvolution across models trained from replicate datasets. b. Fraction of cells (y axis) of each cell type (x axis) obtained after deconvolution with models trained separately by each of three Visium sections and in snRNA-seq. c. Predicted single cells (colored dots, legend) in each Visium voxel (grey circle) based on snRNA-seq data mapped onto Visium section 2 (left) and section 3 (right) (compare to FIG. 3b). Cell assignment within a voxel is random with respect to the specific segmented cell.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to “Deep learning and alignment of spatially-resolved whole transcriptomes of single cells in the mouse brain with Tangram,” by Tommaso Biancalani, Gabriele Scalia, Lorenzo Buffoni, Raghav Avasthi, Ziqing Lu, Aman Sanger, Neriman Tokcan, Charles R. Vanderburg, Asa Segerstolpe, Meng Zhang, Inbal Avraham-Davidi, Sanja Vickovic, Mor Nitzan, Sai Ma, Jason Buenrostro, Nik Bear Brown, Duccio Fanelli, Xiaowei Zhuang, Evan Z. Macosko, Aviv Regev and posted to BioRxiv on August 30, 2020 (bioRxiv 2020.08.29.272831; doi: doi.org/10.1101/2020.08.29.272831). All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide methods of generating spatial maps at single cell resolution. Charting a biological atlas of an organ, such as the brain, requires us to spatially-resolve whole transcriptomes of single cells, and to relate such cellular features to the histological and anatomical scales. Single-cell and single-nucleus RNA-Seq (sc/snRNA-seq) can map cells comprehensively^{5,6}, but relating those to their histological and anatomical positions in the context of an organ's common coordinate framework remains a major challenge and barrier to the construction of a cell atlas⁷⁻¹⁰. Conversely, Spatial Transcriptomics allows for in-situ measurements¹¹⁻¹³ at the histological level, but at lower spatial resolution and with limited sensitivity. Targeted in situ technologies¹⁻³ solve both issues, but are limited in gene throughput which impedes profiling of the entire transcriptome. Finally, as samples are collected for profiling, their registration to anatomical atlases often require human supervision, which is a major obstacle to build pipelines at scale. Here, Applicants demonstrate spatial mapping of cells, histology, and anatomy in the somatomotor area and the visual area of the healthy adult mouse brain. Applicants devise Tangram, a method that aligns snRNA-seq data to various forms of spatial data collected from the same brain region, including MERFISH¹, STARmap², smFISH³, and Spatial Transcriptomics⁴ (Visium), as well as histological images and public atlases. Tangram can map any type of sc/snRNA-seq data, including multi-modal data such as SHARE-seq data⁵, which Applicants used to reveal spatial patterns of chromatin accessibility. Applicants equipped Tangram with a deep learning computer vision pipeline, which allows for automatic identification of anatomical annotations on histological images of mouse brain. By doing so, Tangram reconstructs a genome-wide, anatomically-integrated, spatial map of the visual and somatomotor area with ˜30,000 genes at single-cell resolution, revealing spatial gene expression and chromatin accessibility patterning beyond current limitation of in-situ technologies.

### Example Systems Architectures

Turning now to the drawings, in which like numerals represent like (but not necessarily identical) elements throughout the figures, example embodiments are described in detail.

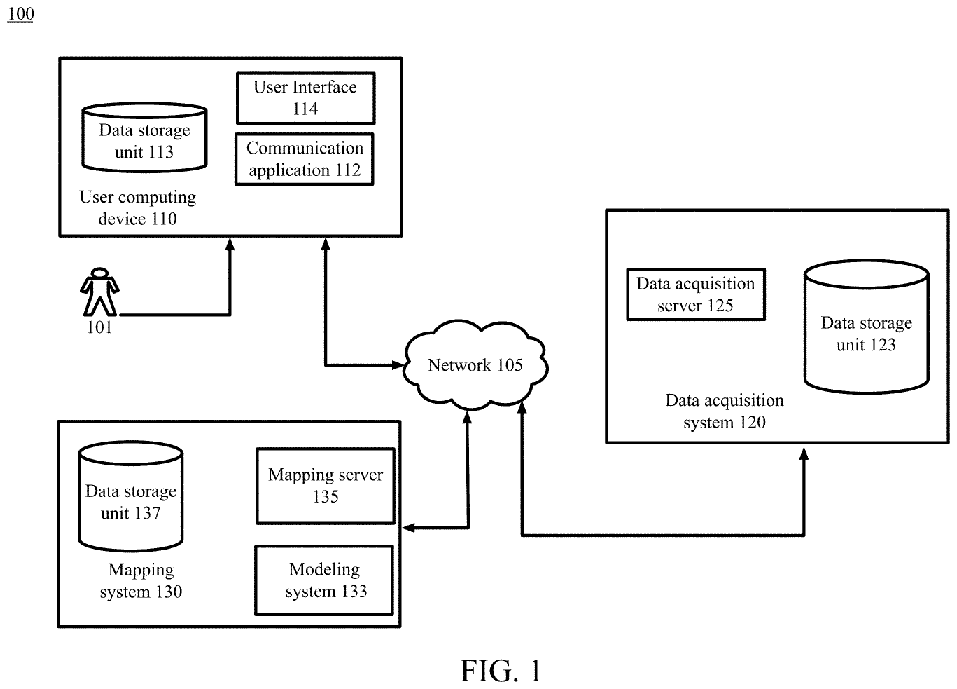

FIG. 1 is a block diagram of depicting a system 100 to learn and align spatially-resolved whole transcriptomes of single cells, in accordance with certain examples. In some embodiments, a user 101 associated with a user computing device 110 must install an application, and/or make a feature selection to obtain the benefits of the techniques described herein.

As depicted in FIG. 1, the system 100 includes network computing devices/systems 110, 120, and 130 that are configured to communicate with one another via one or more networks 105 or via any suitable communication technology.

Each network 105 includes a wired or wireless telecommunication means by which network devices/systems (including devices 110, 120, and 130) can exchange data. For example, each network 105 can include a local area network (“LAN”), a wide area network (“WAN”), an intranet, an Internet, a mobile telephone network, storage area network (“SAN”), personal area network (“PAN”), a metropolitan area network (“MAN”), a wireless local area network (“WLAN”), a virtual private network (“VPN”), a cellular or other mobile communication network, Bluetooth, near field communication (“NFC”), ultra-wideband, or any combination thereof or any other appropriate architecture or system that facilitates the communication of signals and data. Throughout the discussion of example embodiments, it should be understood that the terms “data” and “information” are used interchangeably herein to refer to text, images, audio, video, or any other form of information that can exist in a computer-based environment. The communication technology utilized by the devices/systems 110, 120, and 130 may be similar networks to network 105 or an alternative communication technology.

Each network computing device/system 110, 120, and 130 includes a computing device having a communication module capable of transmitting and receiving data over the network 105 or a similar network. For example, each network device/system 110, 120, and 130 can include a server, desktop computer, laptop computer, tablet computer, a television with one or more processors embedded therein and/or coupled thereto, smartphone, handheld or wearable computer, personal digital assistant (“PDA”), wearable devices such as smartwatches or glasses, or any other wired or wireless, processor-driven device. In the example embodiment depicted in FIG. 1, the network devices/systems 110, 120, and 130 are operated by user 101, data acquisition system operators, and mapping system operators, respectively.

The user computing device 110 includes a user interface 114. The user interface 114 may be used to display a graphical user interface and other information to the user 101 to allow the user 101 to interact with the data acquisition system 120, the mapping system 130, and others. The user interface 114 receives user input for data acquisition and/or mapping and displays results to user 101. In certain examples, the user interface 114 may be provided with a graphical user interface by the data acquisition system 120 and/or the mapping system 130. The user interface 114 may be accessed by the processor of the user computing device 110. The user interface 114 may display a webpage associated with the data acquisition system 120 and/or the mapping system 130. The user interface 114 may be used to provide input, configuration data, and other display directions by the webpage of the data acquisition system 120 and/or the mapping system 130. In certain examples, the user interface 114 may be managed by the data acquisition system 120, the mapping system 130, or others. In certain examples, the user interface 114 may be managed by the user computing device 110 and be prepared and displayed to the user 101 based on the operations of the user computing device 110.

The user 101 can use the communication application 112 on the user computing device 110, which may be, for example, a web browser application or a stand-alone application, to view, download, upload, or otherwise access documents or web pages through the user interface 114 via the network 105. The user computing device 110 can interact with web servers or other computing devices connected to the network 105, including the data acquisition server 125 of the data acquisition system 120 and the mapping server 135 of the mapping system 130. In another example, the user computing device 110 communicates with devices in the data acquisition system 120 and/or the mapping system 130 via NFC or other wireless communication technology, such as Bluetooth, WiFi, infrared, or any other suitable technology.

The user computing device 110 also includes a data storage unit 113 accessible by the user interface 114, the communication application 112, or other applications. The example data storage unit 113 can include one or more tangible computer-readable storage devices. The data storage unit 113 can be stored on the user computing device 110 or can be logically coupled to the user computing device 110. For example, the data storage unit 113 can include on-board flash memory and/or one or more removable memory accounts or removable flash memory. In certain embodiments, the data storage unit 113 may reside in a cloud-based computing system.

An example data acquisition system 120 comprises a data storage unit 123 and an acquisition server 125. The data storage unit 123 can include any local or remote data storage structure accessible to the data acquisition system 120 suitable for storing information. The data storage unit 123 can include one or more tangible computer-readable storage devices, or the data storage unit 123 may be a separate system, such as a different physical or virtual machine or a cloud-based storage service.

In an example embodiment, the data acquisition server 125 communicates with the user computing device 110 and/or the mapping system 130 to transmit requested data. The data may include cell data associated with a specimen from a specific anatomical region or tissue type. The cell data may comprise single cell RNA-seq data (scRNA-seq), single nucleus RNA-seq data (snRNA-seq), single cell ChIP, single cell ATAC-seq, or single cell proteomics. The cell data may be multi-modal single cell data comprising single cell RNA-seq and chromatic accessibility data (SHARE-seq), CITE-seq data, or Patch-seq. The data may also include spatial data from the same specimen as the cell data, or spatial data from the same anatomical region or tissue type from a different specimen than the cell data. In an example, the spatial data may be coarse grained. As used herein, the term “coarse grained” refers to a measure of resolution of the data, such that coarse grain refers to data that has less resolution than single cell resolution and “fine-grained,” which refers to a measure of resolution that is finer than coarse-grained. Thus in certain embodiments, fine-grained refers to single cell resolution and coarse grained refers to spatial data that is less than single cell resolution. For example, a spatial resolution larger than a single cell is considered more coarse and less fine (e.g., coarse may be from 50-100 μm for Spatial Transcriptomics (ST)⁴ or 10 μm for Slide-see). The spatial data may comprise in situ Hybridization (ISH), Single-molecule Fluorescence in situ Hybridization (smFISH), Spatial Transcriptomics (Visium), STARmap, and/or MERFISH.

An example mapping system 130 comprises a modeling system 133, a mapping server 135, and a data storage unit 137. The mapping server 135 communicates with the user computing device 110 and/or the data acquisition system 120 to request and receive data. The data may comprise the data types previously described in reference to the data acquisition server 125.

The modeling system 133 receives an input of data from the mapping server 135. The modeling system 133 can comprise one or more functions to implement an unsupervised deep learning nonlinear optimization program to learn an alignment of the cell data with the spatial data. The unsupervised deep learning nonlinear optimization program may apply one or more similarity functions to the cell and spatial data. In an example, the similarity functions may comprise a Kullback-Liebler (KL) divergence function, a cosine similarity function, a radial basis function (RBF), or any other suitable similarity function. In an alternate example, the unsupervised deep learning nonlinear optimization program may apply one or more spatial correlation functions to learn an alignment of the cell data with the spatial data. Any suitable functions may be applied to learn an alignment of the cell data with the spatial data.

The data storage unit 137 can include any local or remote data storage structure accessible to the data acquisition system 130 suitable for storing information. The data storage unit 137 can include one or more tangible computer-readable storage devices, or the data storage unit 137 may be a separate system, such as a different physical or virtual machine or a cloud-based storage service.

In an alternate embodiment, the functions of either or both of the data acquisition system 120 and the mapping system 130 may be performed by the user computing device 110.

It will be appreciated that the network connections shown are examples, and other means of establishing a communications link between the computers and devices can be used. Moreover, those having ordinary skill in the art having the benefit of the present disclosure will appreciate that the user computing device 110, data acquisition system 120, and the mapping system 130 illustrated in FIG. 1 can have any of several other suitable computer system configurations. For example, a user computing device 110 embodied as a mobile phone or handheld computer may not include all the components described above.

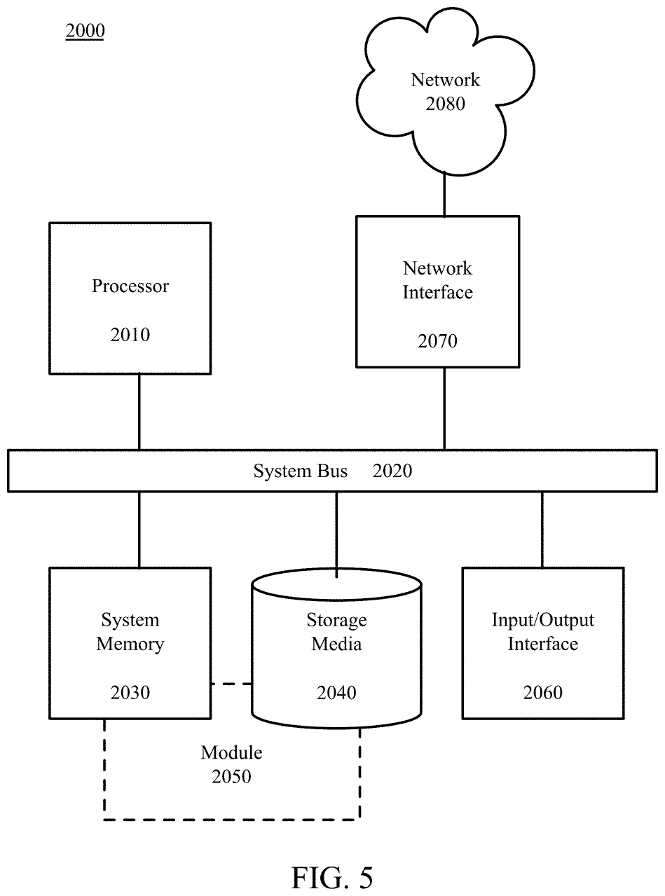

In example embodiments, the network computing devices and any other computing machines associated with the technology presented herein may be any type of computing machine such as, but not limited to, those discussed in more detail with respect to FIG. 5. Furthermore, any modules associated with any of these computing machines, such as modules described herein or any other modules (scripts, web content, software, firmware, or hardware) associated with the technology presented herein may by any of the modules discussed in more detail with respect to FIG. 5. The computing machines discussed herein may communicate with one another as well as other computer machines or communication systems over one or more networks, such as network 105. The network 105 may include any type of data or communications network, including any of the network technology discussed with respect to FIG. 5.

### Example Processes

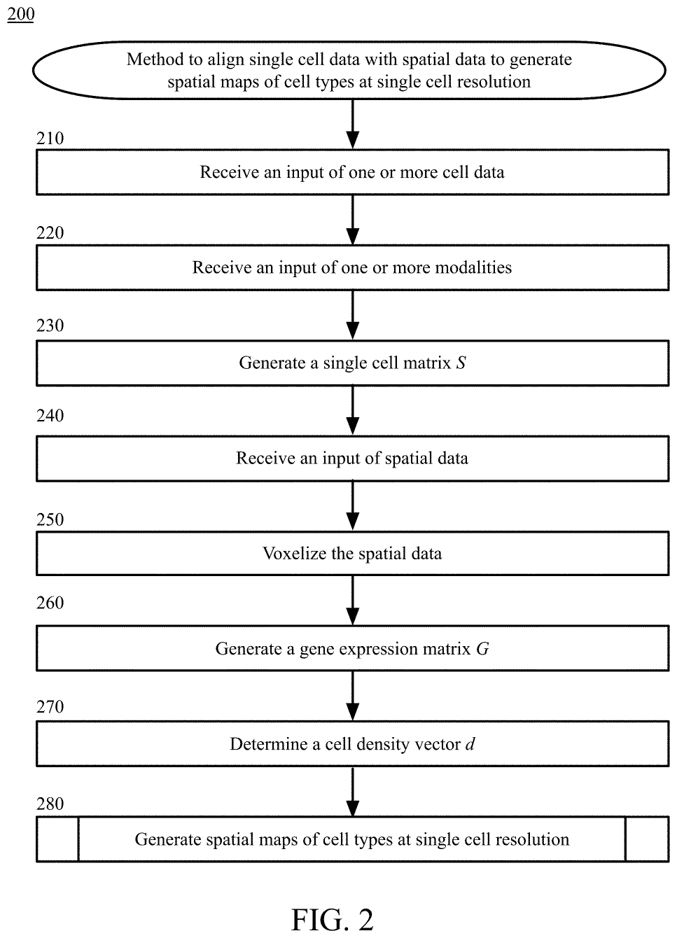

The example methods illustrated in FIGS. 2-4 are described hereinafter with respect to the components of the example architecture 100. The example methods also can be performed with other systems and in other architectures including similar elements.

### Single Cell and Spatial Data Alignment

Referring to FIG. 2, and continuing to refer to FIG. 1 for context, a block flow diagram illustrates methods 200 to align single cell data with spatial data to generate spatial maps of cell types at single cell resolution, in accordance with certain examples of the technology disclosed herein.

In block 210, the mapping system 130 receives an input of one or more cell data to be aligned with spatial data. The mapping system 130 may receive the cell data from the user computing device 110, the data acquisition system 120, or any other suitable source of cell data. The cell data may include cell data associated with a specimen from a specific anatomical region or tissue type. The cell data may comprise single cell RNA-seq data (scRNA-seq), single nucleus RNA-seq data (snRNA-seq), single cell ChIP, single cell ATAC-seq, or single cell proteomics. The cell data may be multi-modal single cell data comprising single cell RNA-seq and chromatic accessibility data (SHARE-seq) or CITE-seq data. Any suitable cell data may be received for alignment.

In block 220, the mapping system 130 receives an input of one or more modalities. The modalities may comprise genes, cell types, or any other modality associated with the cell data. The mapping system 130 may receive the input of one or more modalities from the user computing device 110, the data acquisition system 120, or any other suitable source of modalities. In an example, the one or more modalities may be input into the user computing device 110 by user 101. For simplicity in the following examples, the one or more modalities are genes.

In block 230, the mapping system 130 generates a single cell matrix S. An index of i is assigned to represent cells and an index of k is assigned to genes. The mapping system 130 generates a single cell matrix S with dimensions n_{cell}×n_{genes}, where n_{cells} is the number of single cells in the cell data. Each element Sᵢₖwithin single cell matrix S represents the expression level of gene k in cell i such that each element Sᵢₖ≥0.

In block 240, the mapping system 130 receives an input of spatial data. The mapping system 130 may receive the spatial data from the user computing device 110, the data acquisition system 120, or any other suitable source of spatial data. The spatial data may comprise spatial data from the same specimen as the cell data, or spatial data from the same anatomical region or tissue type from a different specimen than the cell data. In an example, the spatial data may be coarse grained. The spatial data may comprise in situ Hybridization (ISH), Single-molecule Fluorescence in situ Hybridization (smFISH), Spatial Transcriptomics (Visium), STARmap, and/or MERFISH.

In block 250, the mapping system 130 voxelizes the spatial data. A voxel (volume pixel) is a box-shaped component of a three-dimensional image. The mapping system 130 voxelizes the spatial data into spatial voxels at a resolution based on the type of spatial data. For example, if the spatial data is comprised of MERFISH, the resolution may be individual cells. An index of j is assigned to represent the spatial voxels.

In block 260, the mapping system 130 generates a gene expression matrix G. The mapping system 130 generates a gene expression matrix G with dimensions nᵥₒₓₑₗₛ×n_{genes}. Each element Gⱼₖwithin gene expression matrix G represents the expression level of gene k in voxel j such that each element Gⱼₖ≥0.

In block 270, the mapping system 130 determines a cell density vector \vec{d}. The cell density vector \vec{d} is a nᵥₒₓₑₗ-long vector of cell densities, wherein each component dⱼ of vector \vec{d} has a value 0≤dⱼ≤0 and represents the cell density in voxel j. The sum of the components dⱼ of vector \vec{d} is equal to 1 (i.e., Σⱼⁿ^{ᵥₒₓₑₗ} dⱼ=1).

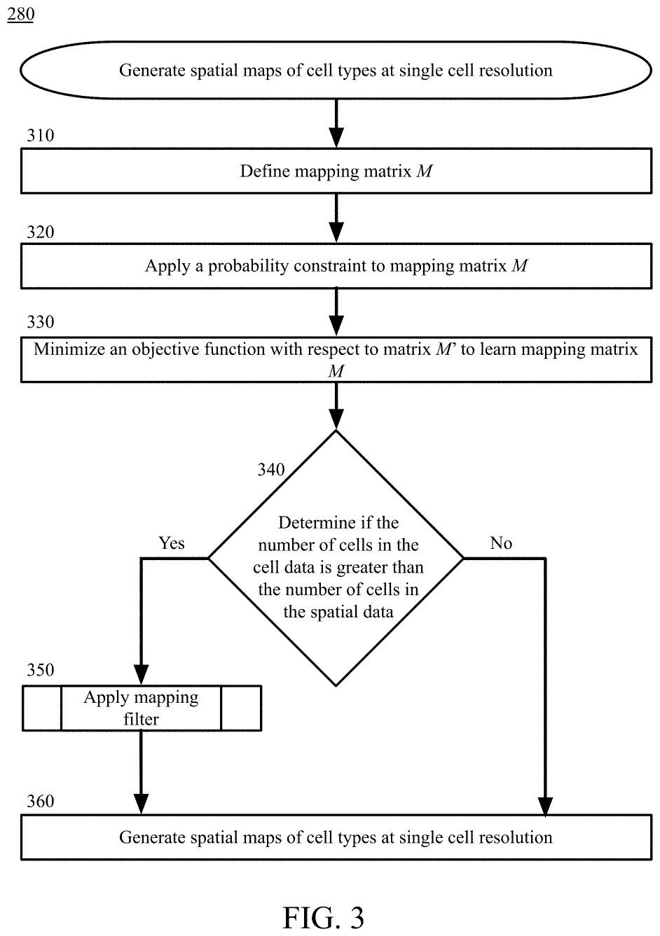

In block 280, the mapping system 130 generates spatial maps of cell types at single cell resolution. Block 280 is described in greater detail in the method 280 of FIG. 3.

### Learn Mapping Matrix M

FIG. 3 is a block flow diagram illustrating methods 280 to generate spatial maps of cell types at single cell resolution, in accordance with certain examples of the technology disclosed herein.

In block 310, the mapping system 130 defines a mapping matrix M. The mapping matrix M has dimensions n_{cells}×nᵥₒₓₑₗₛ. Each element Mᵢⱼ within mapping matrix M represents the probability of cell i being located in voxel j such that each element Mᵢⱼ ≥0.

In block 320, the mapping system 130 applies a probability constraint to mapping matrix M. The probability constraint requires that the elements within mapping matrix M sum to equal 1 (i.e., Σⱼⁿ^{_{vocel}} Mᵢⱼ=1).

In block 330, the mapping system 130 minimizes an objective function with respect to matrix \tilde{M} to learn mapping matrix M . The matrix \tilde{M} is any given matrix with dimensions n_{cells}×nᵥₒₓₑₗₛ. To minimize an objective function with respect to any given matrix \tilde{M}, the following quantities are defined: M^{T}S is defined as the spatial gene expression predicted by the mapping matrix M, and the vector \vec{m} with components mⱼ=Σᵢⁿ^{_{cells}} Mᵢⱼ/n_{cells} is defined as the predicted cell density in voxel j.

A softmax function is applied to matrix \tilde{M}. A softmax function, also known as softargmax or a normalized exponential function, receives a vector or matrix input and normalizes the input values into a probability distribution proportional to the exponentials of the input values. The softmax function is applied to matrix \tilde{M} such that the mapping matrix M has the following elements:

$${M}_{i j} = softmax {( \tilde{M} )}_{i j} = \frac{{e}^{{\tilde{M}}_{ij}}}{{\sum }_{l}^{{n}_{v o x e l s}} {e}^{{\tilde{M}}_{ι l}}} .$$

To learn the mapping matrix M, the following objective function is minimized with respect to matrix \tilde{M}, wherein M=softmax(\tilde{M}): Φ(\tilde{M})=KL(\vec{m}, \vec{d})−Σₖⁿ^{_{genes}}cosₛᵢₘ ((M^{T}S)_{*,k},G_{*,k})−Σⱼⁿ^{ᵥₒₓₑₗₛ}cosₛᵢₘ ((M^{T}S)_{j,*}, G_{j,*}). In the objective function, KL indicates the Kullback-Leibler divergence and cosₛᵢₘ is the cosine similarity function. The first term is the density term where the minimization of the objective function provides a learned density distribution that approaches the expected density distribution. The second term is the gene/voxel expression term where the minimization of the objective function provides a predicted expression for each gene over the voxels that is proportional to the expected gene expression over the voxels. The third term is the voxel/gene expression term where the minimization of the objective function provides a predicted gene expression that is proportional to the expected gene expression. In an example, the minimization of the objective function is achieved through the use of a machine learning algorithm, such as the PyTorch library. The output of the minimization of the objective function is the learned mapping matrix M.

In block 340, the mapping system 130 determines if the number of cells in the cell data is greater than the number of cells in the spatial data. If the number of cells in the cell data is greater than the number of cells in the spatial data, the method proceeds to block 350. If the number of cells in the cell data is not greater than the number of cells in the spatial data, the method proceeds to block 360.

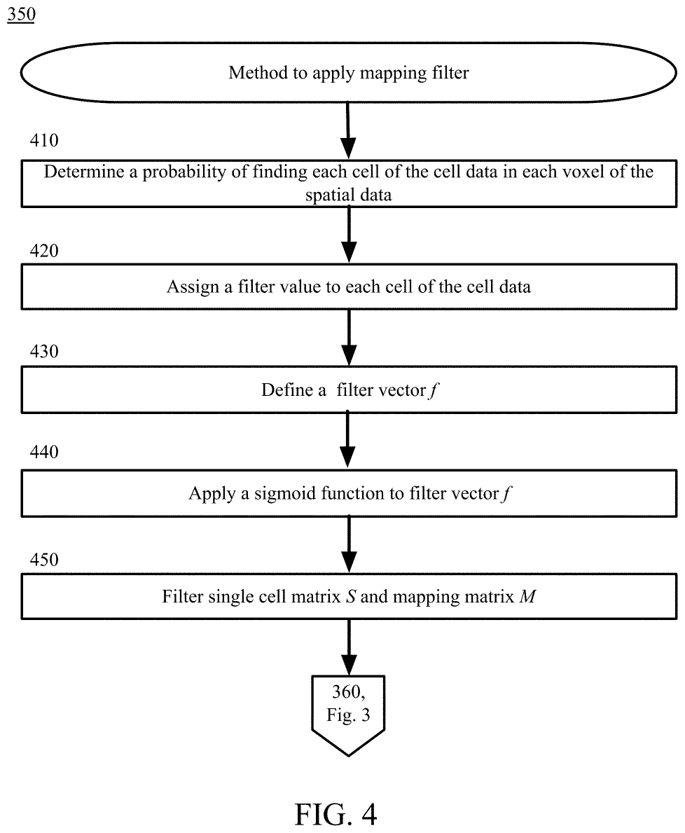

In block 350, the mapping system 130 applies a mapping filter. Block 350 is described in greater detail in the method 350 of FIG. 4.

### Apply a Mapping Filter

FIG. 4 is a block flow diagram depicting methods to apply a mapping filter, in accordance with certain examples of the technology disclosed herein.

In block 410, the mapping system 130 determines a probability of finding each cell of the cell data in each voxel of the spatial data. Referring back to blocks 310 and 330, each element Mᵢⱼ within mapping matrix M represents the probability of cell i being located in voxel j such that each element Mᵢⱼ≥0, and the output of the minimization of the objective function is the learned mapping matrix M. The probability of finding each cell of the cell data in each voxel of the spatial data is represented by each element of the learned mapping matrix M.

In block 420, the mapping system 130 assigns a filter value to each cell of the cell data. The assigned filter value is a Boolean value (i.e., 0's or 1's). A cell is mapped if the filter value is 1 and a cell is not mapped if the filter value is 0. In an example, a filter value of 1 is assigned if the probability of finding a cell of the cell data in the voxel of the spatial data is above a configured threshold. In an example, the configured threshold may be a probability greater than 0.5, 0.75, 0.9, or any suitable probability threshold.

In block 430, the mapping system 130 defines a filter vector \vec{f}. The filter vector \vec{f} has a dimension n_{cells} so that each cell i can either be mapped (fᵢ=1) or not mapped (fᵢ=0).

In block 440, the mapping system 130 applies a sigmoid function to the filter vector \vec{f}. To filter the total number of cells, a count term is utilized. The count term uses a soft constraint such that the number of mapped cells in the filter equals the number of target cells, n_{target_cells}. The mapping system 130 defines a real vector [symbol] with dimension n_{cells} and defines fᵢ=σ( [symbol] ). The sigmoid function a is applied such that the value of fᵢ is 0≤fᵢ≤1. The sigmoid function may be a logistic function, a hyperbolic tangent function, an arctangent function, or any other suitable sigmoid function.

In block 450, the mapping system 130 filters the single cell matrix S and the mapping matrix M . Filtering the single cell matrix S and the mapping matrix M retains the cells that optimally describe the spatial data. To filter the single cell matrix S and the mapping matrix M, the mapping system 130 defines S^{f}=diag(f)·S and M^{f}=diag(f)·M as the filtered versions of the single cell matrix S and the mapping matrix M, respectively. The mapping system 130 defines vector \vec{m}^{f} with components m^{f}ⱼ=Σᵢⁿ^{_{cells}} M^{f}/Σᵢⁿ^{_{cells}} fᵢ, as the predicted density of filtered cells in voxel j. To determine S^{f} and M^{f}, the mapping system 130 minimizes the following objective function with respect to \tilde{M} and \tilde{\vec{f}}: Φ(\tilde{M}, \tilde{\vec{f}})=KL(\vec{m}^{f}, \vec{d})−τₖⁿ^{_{genes}} cosₛᵢₘ ((M^{T}S^{f})_{*,k}, G_{*,k})−Σⱼⁿ^{ᵥₒₓₑₗₛ} cosₛᵢₘ((M^{T}S^{f})_{j,*}, G_{j,*})−λᵣ_{₁} Σ_{i,j}ⁿ^{_{cells,}} ⁿ^{ᵥₒₓₑₗₛ} Mᵢⱼ log (Mᵢⱼ)+abs(Σᵢⁿ^{_{cells}} fᵢ−n_{target_cells})+Σᵢⁿ^{_{cells}}(fᵢ−fᵢ²). In an example, the minimization of the objective function is achieved through the use of a machine learning algorithm, such as the PyTorch library. From block 450, the method 350 returns to FIG. 3 block 360.

### Generate Spatial Maps at Single Cell Resolution

In block 360, the mapping system 130 generates spatial maps of cell types at single cell resolution. The mapping system 130 defines an annotation matrix A as a matrix with dimensions n_{cells}×nₐₙₙₒₜₐₜᵢₒₙₛ, where nₐₙₙₒₜₐₜᵢₒₙₛ is the number of different annotations characterizing single cells, for example genes, cell types, or any other suitable modality. The mapping system 130 generates a spatial map by computing: A_{transf}=M^{T}A. If a mapping filter has been applied, the mapping system 130 generates a spatial map by computing: A^{f}_{transf}=M^{T} (diag(f)·A). The computed matrix A_{transf} or A_{transf}^{f} has dimensions nᵥₒₓₑₗ×nₐₙₙₒₜₐₜᵢₒₙₛ and represents a spatial map of the annotation in space.

### Other Example Embodiments

FIG. 5 depicts a computing machine 2000 and a module 2050 in accordance with certain examples. The computing machine 2000 may correspond to any of the various computers, servers, mobile devices, embedded systems, or computing systems presented herein. The module 2050 may comprise one or more hardware or software elements configured to facilitate the computing machine 2000 in performing the various methods and processing functions presented herein. The computing machine 2000 may include various internal or attached components such as a processor 2010, system bus 2020, system memory 2030, storage media 2040, input/output interface 2060, and a network interface 2070 for communicating with a network 2080.

The computing machine 2000 may be implemented as a conventional computer system, an embedded controller, a laptop, a server, a mobile device, a smartphone, a set-top box, a kiosk, a router or other network node, a vehicular information system, one or more processors associated with a television, a customized machine, any other hardware platform, or any combination or multiplicity thereof. The computing machine 2000 may be a distributed system configured to function using multiple computing machines interconnected via a data network or bus system.

The processor 2010 may be configured to execute code or instructions to perform the operations and functionality described herein, manage request flow and address mappings, and to perform calculations and generate commands. The processor 2010 may be configured to monitor and control the operation of the components in the computing machine 2000. The processor 2010 may be a general purpose processor, a processor core, a multiprocessor, a reconfigurable processor, a microcontroller, a digital signal processor (“DSP”), an application specific integrated circuit (“ASIC”), a graphics processing unit (“GPU”), a field programmable gate array (“FPGA”), a programmable logic device (“PLD”), a controller, a state machine, gated logic, discrete hardware components, any other processing unit, or any combination or multiplicity thereof. The processor 2010 may be a single processing unit, multiple processing units, a single processing core, multiple processing cores, special purpose processing cores, co-processors, or any combination thereof. According to certain examples, the processor 2010 along with other components of the computing machine 2000 may be a virtualized computing machine executing within one or more other computing machines.

The system memory 2030 may include non-volatile memories such as read-only memory (“ROM”), programmable read-only memory (“PROM”), erasable programmable read-only memory (“EPROM”), flash memory, or any other device capable of storing program instructions or data with or without applied power. The system memory 2030 may also include volatile memories such as random-access memory (“RAM”), static random-access memory (“SRAM”), dynamic random-access memory (“DRAM”), and synchronous dynamic random-access memory (“SDRAM”). Other types of RAM also may be used to implement the system memory 2030. The system memory 2030 may be implemented using a single memory module or multiple memory modules. While the system memory 2030 is depicted as being part of the computing machine 2000, one skilled in the art will recognize that the system memory 2030 may be separate from the computing machine 2000 without departing from the scope of the subject technology. It should also be appreciated that the system memory 2030 may include, or operate in conjunction with, a non-volatile storage device such as the storage media 2040.

The storage media 2040 may include a hard disk, a floppy disk, a compact disc read only memory (“CD-ROM”), a digital versatile disc (“DVD”), a Blu-ray disc, a magnetic tape, a flash memory, other non-volatile memory device, a solid state drive (“SSD”), any magnetic storage device, any optical storage device, any electrical storage device, any semiconductor storage device, any physical-based storage device, any other data storage device, or any combination or multiplicity thereof. The storage media 2040 may store one or more operating systems, application programs and program modules such as module 2050, data, or any other information. The storage media 2040 may be part of, or connected to, the computing machine 2000. The storage media 2040 may also be part of one or more other computing machines that are in communication with the computing machine 2000 such as servers, database servers, cloud storage, network attached storage, and so forth.

The module 2050 may comprise one or more hardware or software elements configured to facilitate the computing machine 2000 with performing the various methods and processing functions presented herein. The module 2050 may include one or more sequences of instructions stored as software or firmware in association with the system memory 2030, the storage media 2040, or both. The storage media 2040 may therefore represent examples of machine or computer readable media on which instructions or code may be stored for execution by the processor 2010. Machine or computer readable media may generally refer to any medium or media used to provide instructions to the processor 2010. Such machine or computer readable media associated with the module 2050 may comprise a computer software product. It should be appreciated that a computer software product comprising the module 2050 may also be associated with one or more processes or methods for delivering the module 2050 to the computing machine 2000 via the network 2080, any signal-bearing medium, or any other communication or delivery technology. The module 2050 may also comprise hardware circuits or information for configuring hardware circuits such as microcode or configuration information for an FPGA or other PLD.

The input/output (“I/O”) interface 2060 may be configured to couple to one or more external devices, to receive data from the one or more external devices, and to send data to the one or more external devices. Such external devices along with the various internal devices may also be known as peripheral devices. The I/O interface 2060 may include both electrical and physical connections for coupling in operation the various peripheral devices to the computing machine 2000 or the processor 2010. The I/O interface 2060 may be configured to communicate data, addresses, and control signals between the peripheral devices, the computing machine 2000, or the processor 2010. The I/O interface 2060 may be configured to implement any standard interface, such as small computer system interface (“SCSI”), serial-attached SCSI (“SAS”), fiber channel, peripheral component interconnect (“PCP”), PCI express (PCIe), serial bus, parallel bus, advanced technology attached (“ATA”), serial ATA (“SATA”), universal serial bus (“USB”), Thunderbolt, FireWire, various video buses, and the like. The I/O interface 2060 may be configured to implement only one interface or bus technology. Alternatively, the I/O interface 2060 may be configured to implement multiple interfaces or bus technologies. The I/O interface 2060 may be configured as part of, all of, or to operate in conjunction with, the system bus 2020. The I/O interface 2060 may include one or more buffers for buffering transmissions between one or more external devices, internal devices, the computing machine 2000, or the processor 2010.

The I/O interface 2060 may couple the computing machine 2000 to various input devices including mice, touch-screens, scanners, electronic digitizers, sensors, receivers, touchpads, trackballs, cameras, microphones, keyboards, any other pointing devices, or any combinations thereof. The I/O interface 2060 may couple the computing machine 2000 to various output devices including video displays, speakers, printers, projectors, tactile feedback devices, automation control, robotic components, actuators, motors, fans, solenoids, valves, pumps, transmitters, signal emitters, lights, and so forth.

The computing machine 2000 may operate in a networked environment using logical connections through the network interface 2070 to one or more other systems or computing machines across the network 2080. The network 2080 may include wide area networks (WAN), local area networks (LAN), intranets, the Internet, wireless access networks, wired networks, mobile networks, telephone networks, optical networks, or combinations thereof. The network 2080 may be packet switched, circuit switched, of any topology, and may use any communication protocol. Communication links within the network 2080 may involve various digital or analog communication media such as fiber optic cables, free-space optics, waveguides, electrical conductors, wireless links, antennas, radio-frequency communications, and so forth.

The processor 2010 may be connected to the other elements of the computing machine 2000 or the various peripherals discussed herein through the system bus 2020. It should be appreciated that the system bus 2020 may be within the processor 2010, outside the processor 2010, or both. According to certain examples, any of the processor 2010, the other elements of the computing machine 2000, or the various peripherals discussed herein may be integrated into a single device such as a system on chip (“SOC”), system on package (“SOP”), or ASIC device.

Examples may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing examples in computer programming, and the examples should not be construed as limited to any one set of computer program instructions. Further, a skilled programmer would be able to write such a computer program to implement an example of the disclosed examples based on the appended flow charts and associated description in the application text. Therefore, disclosure of a particular set of program code instructions is not considered necessary for an adequate understanding of how to make and use examples. Further, those ordinarily skilled in the art will appreciate that one or more aspects of examples described herein may be performed by hardware, software, or a combination thereof, as may be embodied in one or more computing systems. Moreover, any reference to an act being performed by a computer should not be construed as being performed by a single computer as more than one computer may perform the act.

The examples described herein can be used with computer hardware and software that perform the methods and processing functions described herein. The systems, methods, and procedures described herein can be embodied in a programmable computer, computer-executable software, or digital circuitry. The software can be stored on computer-readable media. For example, computer-readable media can include a floppy disk, RAM, ROM, hard disk, removable media, flash memory, memory stick, optical media, magneto-optical media, CD-ROM, etc. Digital circuitry can include integrated circuits, gate arrays, building block logic, field programmable gate arrays (FPGA), etc.

The example systems, methods, and acts described in the examples presented previously are illustrative, and, in alternative examples, certain acts can be performed in a different order, in parallel with one another, omitted entirely, and/or combined between different examples, and/or certain additional acts can be performed, without departing from the scope and spirit of various examples. Accordingly, such alternative examples are included in the scope of the following claims, which are to be accorded the broadest interpretation to encompass such alternate examples.

### Spatial and Single Cell Data

The present invention can utilize any single cell and spatial data to generate the output spatial data having single cell resolution. The single cell and spatial data must share at least one feature. As used herein, “feature” refers to any molecular biomarker that can be assayed in single cells and in spatially resolved data. For example, transcriptome, chromatin accessibility, epigenetic data, or any combination thereof. Thus, the shared feature can be a shared expression of one or more genes, a shared region of chromatin accessibility, or a shared pattern of chromatin modification at a specific genomic locus. In certain embodiments, the data is generated by the user or is obtained by the user from another source. The shared features can be as low as 10 shared features (e.g., Applicants demonstrate the use of 22 features, see, examples). Preferably, each cell type that is to be mapped has at least 1 feature that can be used by the system to align the single cell data to the spatial data. In one embodiments each cell type has a unique expression or presentation of the feature, such that less features are required. If features used are present in multiple cell types, then alignment may require determining patterns of features in each cell type. Thus, more features would be required for alignment. In certain embodiments, the spatial data shares at least 10, 20, 30, 40, 50, 60, 70, 80 ,90, 100, 200, 300, 400, or more than 500 features. The single cell data generally has more measured features than the spatial data and can include up to a genome wide set of features, such as expression of every gene in the cell.

### Cell Atlas

In certain embodiments, the data can be obtained from a single cell atlas or cell atlas. As used herein “single cell atlas” refers to any collection of single cell data from any tissue sample of interest having a phenotype of interest (see, e.g., Rozenblatt-Rosen O, Stubbington M J T, Regev A, Teichmann S A., The Human Cell Atlas: from vision to reality., Nature. 2017 Oct. 18; 550(7677):451-453; and Regev, A. et al. The Human Cell Atlas Preprint available at bioRxiv at dx.doi.org/10.1101/121202 (2017)). In preferred embodiments, single cell data is obtained from one or more tissue samples, more preferably, one or more tissue samples from one or more subjects. The subjects preferably include one or more subjects having a phenotype and one or more control subjects. The single cell data can include, but is not limited to transcriptome, chromatin accessibility, epigenetic data, or any combination thereof. A single cell atlas can refer to any collection of single cell data from any tissue sample. The number of cells analysed in the atlas may be about 1,000, 2,000, 5,000, 10,000, 20,000, 50,000, 100,000, 500,000, or more than a million cells. The single cell atlas can also include biological and medical information for the subjects where the tissue samples were obtained.

A single cell atlas for a tissue may be constructed by measuring single cell transcriptomes. The single cell atlas can be used as a roadmap for any phenotype present in or associated with a specific tissue (e.g., a “Google Map” of patient tissue samples). The atlas can be generated by providing: (1) biological information, including medical records, histology, single cell profiles, and genetic information, and (2) data, including multiplexed ion beam imaging (MIBI) (see, e.g., Angelo et al., Nat Med. 2014 April; 20(4): 436-442), NanoString (DSP, digital spatial profiling) (see e.g., Geiss G K, et al., Direct multiplexed measurement of gene expression with color-coded probe pairs. Nat Biotechnol. 2008 March; 26(3):317-25), microbiome, immunoprofiling, and sequencing (e.g., bulk and single cell sequencing). Pathology of tissue samples can be performed. Tissue samples can be dissociated for scRNA-seq, flow cytometry and cell culture. Tissues can also be snap frozen for analysis of DNA by WES, bulk RNA-seq, and epigenetics. Tissue can also be OCT frozen for multiplex imaging. The data obtained can be computationally analyzed.

Non-limiting examples of a single cell atlas applicable to the present invention are disclosed in U.S. Ser. No. 16/072,674, WO 2018/191520, WO 2018/191558, U.S. Ser. No. 16/348,911, WO 2019/018440, U.S. Ser. No. 15/844,601, and U.S. Ser. No. 62/888,347. See, also, Darmanis, S. et al. Proc. Natl Acad. Sci. USA 112, 7285-7290 (2015); Lake, B. B. et al. Science 352, 1586-1590 (2016); Pollen, A. A. et al. Nature Biotechnol. 32, 1053-1058 (2014); Tasic, B. et al. Nature Neurosci. 19, 335-346 (2016); Zeisel, A. et al. Science 347, 1138-1142 (2015); Grun. D. et al Nature 525, 251-255 (2015); Shekhar, K. et al. Cell 166, 1308-1323 (2016); Villani, A. C. et al. Science 356, eaah4573 (2017); Lonnberg, T. et al. Sci. Immunol. 2, eaa12192 (2017); Tirosh, I. et al. Science 352, 189-196 (2016); Venteicher A S, et al., Decoupling genetics, lineages, and microenvironment in IDH-mutant gliomas by single-cell RNA-seq., Science. 2017 Mar. 31; 355(6332); Tirosh, I. et al. Single-cell RNA-seq supports a developmental hierarchy in human oligodendroglioma. Nature. 2016 Nov. 10; 539(7628):309-313; Drokhlyansky et al., The enteric nervous system of the human and mouse colon at a single-cell resolution. bioRxiv 746743; doi: doi.org/10.1101/746743; Smillie C S. et al., Intra- and Inter-cellular Rewiring of the Human Colon during Ulcerative Colitis. Cell. 2019 Jul. 25; 178(3):714-730.e22; Montoro D T. et al., A revised airway epithelial hierarchy includes CFTR-expressing ionocytes. Nature. 2018 August; 560(7718):319-324; and Haber A L, et al., A single-cell survey of the small intestinal epithelium. Nature. 2017 Nov. 16; 551(7680):333-339. Smillie et al. shows a cell atlas of UC, a complex disease atlas. Smillie et al. further shows that the atlas can be built from involved and uninvolved tissue in patients, in comparison to the healthy reference from a human cell atlas. A relatively small number of individuals provides a robust catalog (i.e., atlas).

In certain embodiments, single cell transcriptomes are included in the cell atlas. As used herein the term “transcriptome” refers to the set of transcripts molecules. In some embodiments, transcript refers to RNA molecules, e.g., messenger RNA (mRNA) molecules, small interfering RNA (siRNA) molecules, transfer RNA (tRNA) molecules, ribosomal RNA (rRNA) molecules, and complimentary sequences, e.g., cDNA molecules. In some embodiments, a transcriptome refers to a set of mRNA molecules. In some embodiments, a transcriptome refers to a set of cDNA molecules. In some embodiments, a transcriptome refers to one or more of mRNA molecules, siRNA molecules, tRNA molecules, rRNA molecules, in a sample, for example, a single cell or a population of cells. In some embodiments, a transcriptome refers to cDNA generated from one or more of mRNA molecules, siRNA molecules, tRNA molecules, rRNA molecules, in a sample, for example, a single cell or a population of cells. In some embodiments, a transcriptome refers to 50%, 55, 60, 65, 70, 75, 80, 85, 90, 91, 92, 93, 94, 95, 96, 97, 98, 99, 99.9, or 100% of transcripts from a single cell or a population of cells. In some embodiments, transcriptome not only refers to the species of transcripts, such as mRNA species, but also the amount of each species in the sample. In some embodiments, a transcriptome includes each mRNA molecule in the sample, such as all the mRNA molecules in a single cell.

### Single Cell Sequencing

In certain embodiments, the single cell data can be generated using single cell sequencing or single nuclei sequencing. In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as W02016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

### Single Nuclei Sequencing

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; International patent application number PCT/US2016/059239, published as WO2017164936 on Sep. 28, 2017; International patent application number PCT/US2018/060860, published as WO/2019/094984 on May 16, 2019; International patent application number PCT/US2019/055894, published as WO/2020/077236 on Apr. 16, 2020; and Drokhlyansky, et al., “The enteric nervous system of the human and mouse colon at a single-cell resolution,” bioRxiv 746743; doi: doi.org/10.1101/746743, which are herein incorporated by reference in their entirety.

### Single Cell Chromatin Accessibility

In certain embodiments, the invention involves the Assay for Transposase Accessible Chromatin using sequencing (ATAC-seq) as described. (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US20160208323A1; US20160060691A1; and WO2017156336A1).

### Single Cell Chip

In certain embodiments, epigenetic features can be spatially mapped. In certain embodiments, genome wide chromatin immunoprecipitation is used (ChIP) (see, e.g., Rotem, et al., Single-cell ChIP-seq reveals cell subpopulations defined by chromatin state, Nat Biotechnol 33,1165-1172 (2015)). In certain embodiments, ChIP data can be compared to orthogonal single-cell gene expression data. The single cell sequencing data can then be used to align chromatin signatures to spatial data.

### Single Cell HiC

In certain embodiments, epigenetic features can be chromatin contact domains, chromatin loops, superloops, or chromatin architecture data, such as obtained by single cell HiC (see, e.g., Rao et al., Cell. 2014 Dec. 18; 159(7):1665-80; and Ramani, et al., Sci-Hi-C: A single-cell Hi-C method for mapping 3D genome organization in large number of single cells Methods. 2020 Jan. 1; 170: 61-68).

### Single Cell Proteomics

In certain embodiments, single cell proteomics can be used to generate the single cell data. In certain embodiments, the single cell proteomics data is combined with single cell transcriptome data. Non-limiting examples include multiplex analysis of single cell constituents (US20180340939A), single-cell proteomic assay using aptamers (US20180320224A1), and methods of identifying multiple epitopes in cells (US20170321251A1).

### Multimodal Technology

In certain embodiments, SHARE-Seg⁵ is used to generate single cell RNA-seq and chromatin accessibility data. In certain embodiments, CITE-see (cellular proteins) is used to generate single cell RNA-seq and proteomics data. In certain embodiments, Patch-see is used to generate single cell RNA-seq and patch-clamping electrophysiological recording and morphological analysis of single neurons data (e.g., for the brain or enteric nervous system (ENS)) (see, e.g., van den Hurk, et al., Patch-Seq Protocol to Analyze the Electrophysiology, Morphology and Transcriptome of Whole Single Neurons Derived From Human Pluripotent Stem Cells, Front Mol Neurosci. 2018; 11: 261).

### Spatial Data Technology

The spatial data used in the present invention can be any spatial data. Methods of generating spatial data of varying resolution are known in the art, for example, ISS¹⁶, MERFISH¹, smFISH³, osmFISH¹⁷, STARMap², Targeted ExSeq¹⁸, seqFISH+¹⁹, Spatial Transcriptomics methods (e.g., Spatial Transcriptomics (ST)⁴ (now available commercially as Visium), Slide-seq²⁰, or High Definition Spatial Trascriptomics²¹. In certain embodiments, proteomics and spatial patterning using antenna networks is used to spatially map a tissue specimen and this data can be further used to align single cell data to a larger tissue specimen (see, e.g., US20190285644A1). In certain embodiments, the spatial data can be immunohistochemistry data or immunofluorescence data.

### Use of Shared Features

The spatial methods described herein share features with the single cell methods in order to generate the single cell resolution maps of the present invention. In certain embodiments, gene expression or transcriptome data (e.g., genes) can be shared between the single cell data and spatial data. Shared genes can advantageously allow multimodal data that includes gene expression to be spatially aligned. For example chromatin accessibility data, proteomics data, patch-clamp and morphological data can be spatially aligned to single cells. Shared regions of accessible chromatin can be used to spatially align genome wide accessibility to a specimen. Shared regions of epigenetic marks can be used to spatially align genome wide chromatin modifications to a specimen. In certain embodiments, multiple single-Chip data sets for different markers may be used to determine spatially resolved chromatin patterns. In certain embodiments, specific regions of a genome may be labeled in spatial data and chromatin accessibility or epigenetic marks determined for those regions. These shared regions with single cell data can be used to generate a genome wide spatially resolved map. In certain embodiments, CITE-seq can be used to align spatial data that includes gene expression and/or surface labeled proteins. For example, CITE-seq can be used to align transcriptome data to spatially data that only includes surface marker labeling.

### Histology

The methods and systems provided herein can also be used to align single cell resolution spatial maps with histological samples. In certain embodiments, only a few features need to be determined by histological methods because those features will be shared with a genome wide spatially resolved map of the present invention, such that the system can align single cells to the histological data (e.g., 1, 2, 3, 4, 5, 6, 7, 8, 9 or 10 features). For example, a spatial map can be used to bridge single cell data to a histological sample of the same tissue or anatomical region. In certain embodiments, the spatial map is registered on an anatomically annotated common coordinate framework (CCF) (see, e.g., Wang, et al., The Allen Mouse Brain Common Coordinate Framework: A 3D Reference Atlas, Cell. 2020 May 14; 181(4):936-953.e20; Lein E, et al. Genome-wide atlas of gene expression in the adult mouse brain. Nature, 2007; 445:168-76; and Allen Mouse Brain Atlas: mouse.brain-map.org/). As used herein, an “anatomically annotated common coordinate framework” refers to a common reference map that uniquely and reproducibly defines any location in an organism (e.g., human) (see, e.g., Rood, et al., Toward a Common Coordinate Framework for the Human Body, Cell, 2019 Dec. 12; 179(7):1455-1467). A CCF may use coordinate systems and the use of common landmarks for the integration of reference maps at differing scales in one common framework. The CCF is preferably a 3D framework.

Histology, also known as microscopic anatomy or microanatomy, is the branch of biology which studies the microscopic anatomy of biological tissues. Histology is the microscopic counterpart to gross anatomy, which looks at larger structures visible without a microscope. Although one may divide microscopic anatomy into organology, the study of organs, histology, the study of tissues, and cytology, the study of cells, modern usage places these topics under the field of histology. In medicine, histopathology is the branch of histology that includes the microscopic identification and study of diseased tissue. Biological tissue has little inherent contrast in either the light or electron microscope. Staining is employed to give both contrast to the tissue as well as highlighting particular features of interest. When the stain is used to target a specific chemical component of the tissue (and not the general structure), the term histochemistry is used. Antibodies can be used to specifically visualize proteins, carbohydrates, and lipids. This process is called immunohistochemistry, or when the stain is a fluorescent molecule, immunofluorescence. This technique has greatly increased the ability to identify categories of cells under a microscope. Other advanced techniques, such as nonradioactive in situ hybridization, can be combined with immunochemistry to identify specific DNA or RNA molecules with fluorescent probes or tags that can be used for immunofluorescence and enzyme-linked fluorescence amplification.

Although specific examples have been described above in detail, the description is merely for purposes of illustration. It should be appreciated, therefore, that many aspects described above are not intended as required or essential elements unless explicitly stated otherwise. Modifications of, and equivalent components or acts corresponding to, the disclosed aspects of the examples, in addition to those described above, can be made by a person of ordinary skill in the art, having the benefit of the present disclosure, without departing from the spirit and scope of examples defined in the following claims, the scope of which is to be accorded the broadest interpretation so as to encompass such modifications and equivalent structures.

Further embodiments are illustrated in the following Examples which are given for illustrative purposes only and are not intended to limit the scope of the invention.

### EXAMPLES

### Example 1—Tangram: Learning of Spatially-Resolved Single Cell Transcriptomes by Alignment

Here, Applicants present Tangram, a deep learning framework to address both challenges: learn spatial gene expression maps transcriptome-wide at single cell resolution, and relate those to histological and anatomical information from the same specimens. Tangram learns a spatial alignment of sc/snRNA-seq data from a reference spatial data of any kind—either fine or coarse grained, as Applicants demonstrate by spatially mapping snRNA-seq data from the isocortex of the adult healthy mouse brain using each of five kinds of spatial supports, at different levels of resolution and gene coverage: ISH, smFISH, Visium (Spatial Transcriptomics), STARmap and MERFISH. Tangram produces consistent spatial maps of cell types, overcomes limitations in throughput or resolution, corrects low-quality genes, even in high resolution methods, provides single cell resolution for low-resolution methods, and provides genome-wide coverage for targeted methods. By mapping multi-modal single data (SHARE-seg⁵) on spatial support, Tangram visualizes spatial patterns of chromatin accessibility and transcription factor motif scores at single cell resolution. Finally, Tangram includes a dedicated novel computer vision module that leverages histological data, and maps it to anatomical positions in an existing Common Coordinate Framework in the brain. If a histology image is available, even without any further annotation, this module relates all scales, to a single integrated atlas.

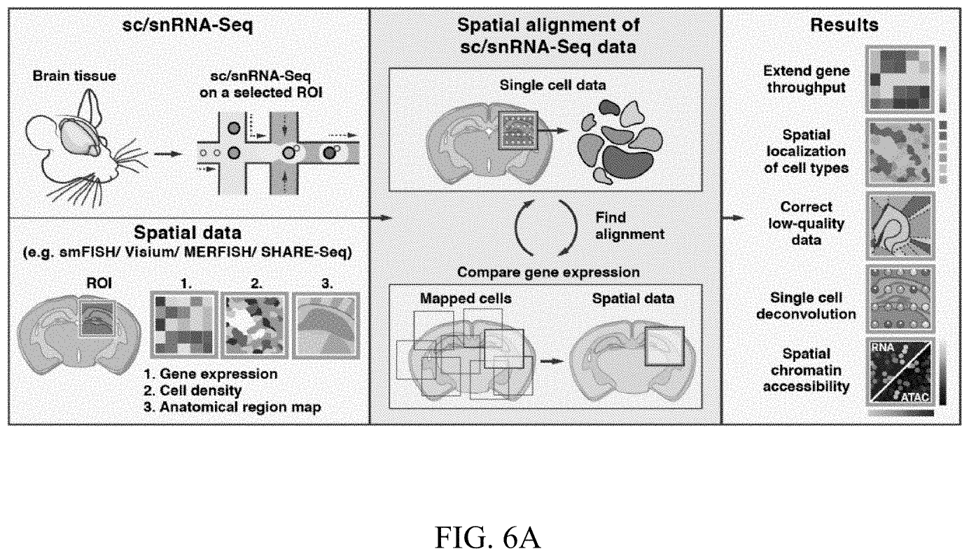

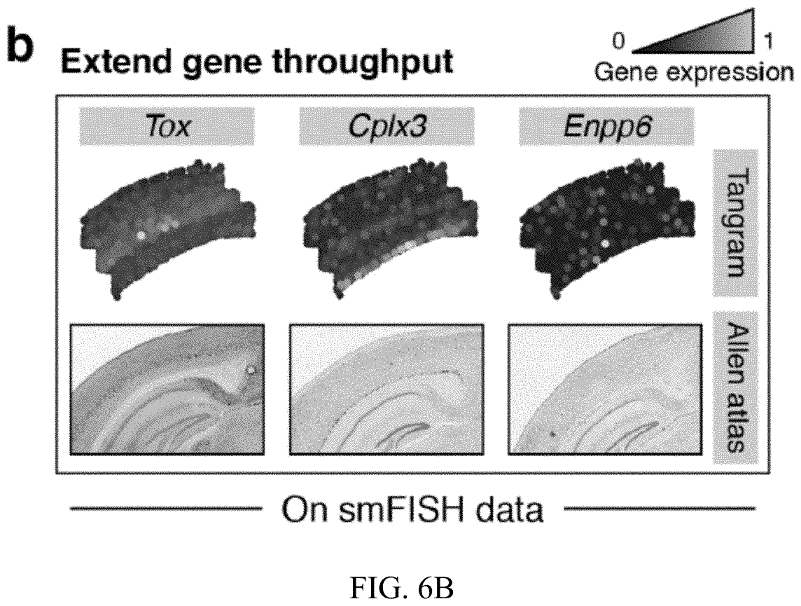

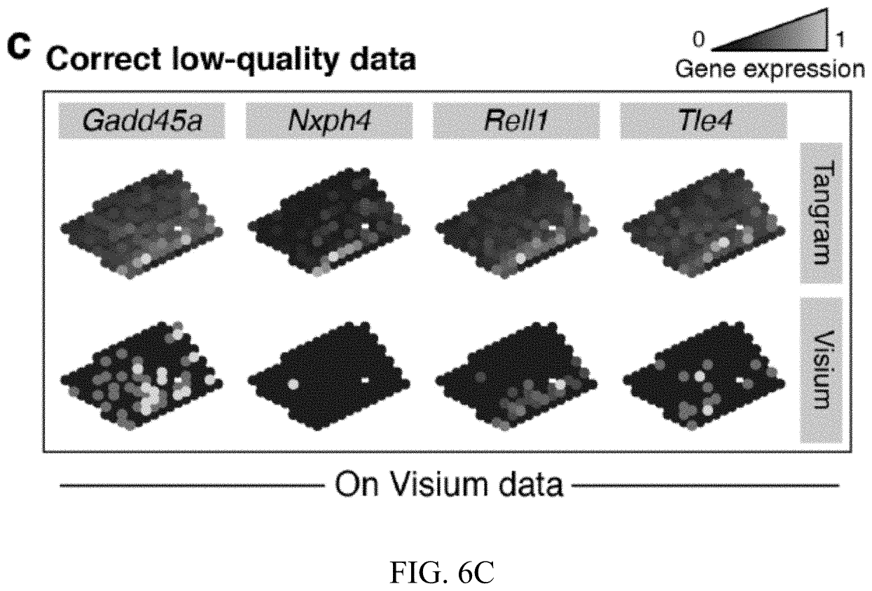

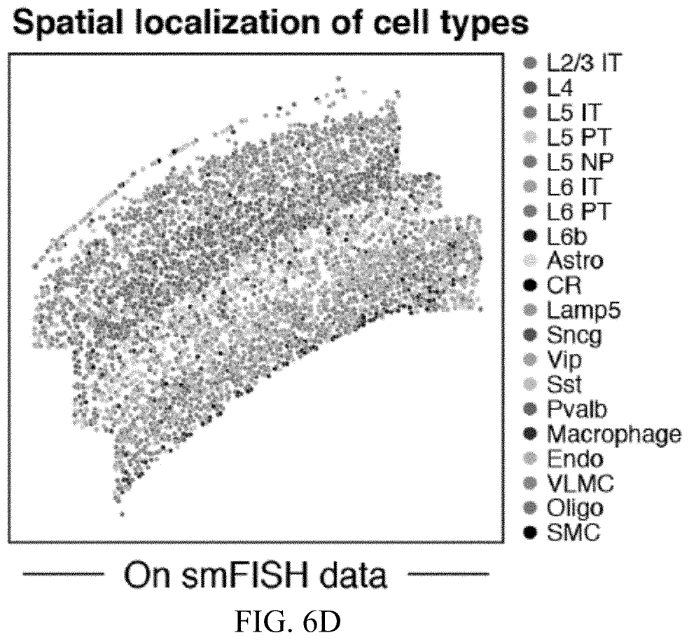

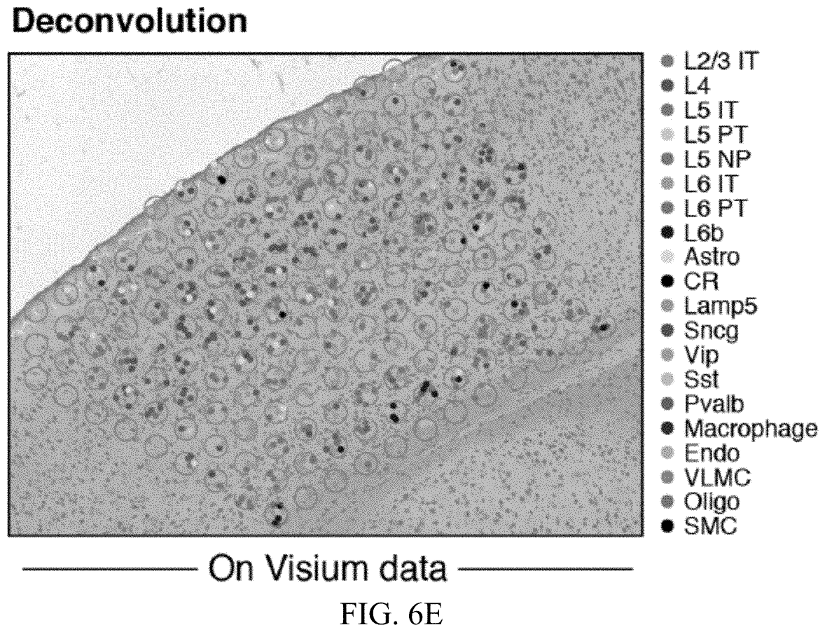

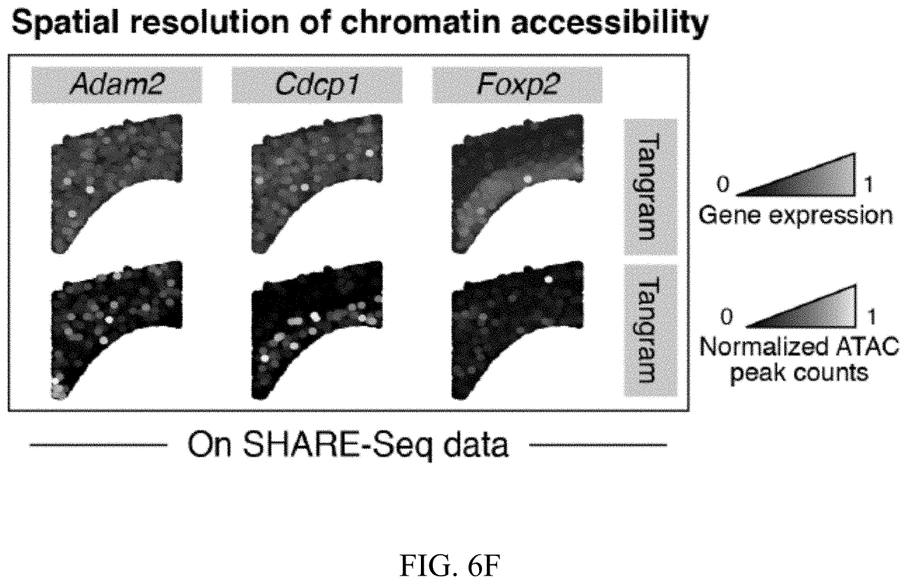

Applicants developed Tangram, an algorithm that uses sc/snRNA-seq as “puzzle pieces” to align in space to match “the shape” of the spatial data (FIG. 6a). The input to Tangram is sc/snRNA-seq data along with spatial data from the same region or tissue type, from any currently available spatial method (e.g., MERFISH, smFISH, STARmap, ISH, or Visium), requiring only that the two modalities share at least some subset of common genes. Intuitively, Tangram first randomly places the sc/snRNA-seq profiles in space, then computes an objective function that mimics the spatial correlation between each gene in the sc/snRNA-seq data and in the spatial data. Tangram then rearranges the sc/snRNA-seq profiles in space to maximize the total spatial correlation across the genes shared by the datasets. When Tangram finishes, the mapped sc/snRNA-seq profiles constitute the new spatial data: they now contain all genes at single cell resolution and with spatial position. From the learned mapping function, Tangram can (1) expand from a measured subset of genes to genome wide profiles (FIG. 6b); (2) correct low-quality spatial measurements (FIG. 6c); (3) map the location of cells of different types (FIG. 6d); (4) deconvolve low resolution measurements to single cells (FIGS. 6e); and (5) resolve spatial patterns of chromatin accessibility at single-cell resolution by aligning multi-modal data (FIG. 6e).

Technically, Tangram is based on non-convex optimization (for full details, see Methods), where the Tangram optimization function rewards the spatial alignment of sc/snRNA-seq data using two similarity functions: cell density distributions are compared using Kullback-Leibler (KL) divergence, whereas gene expression is assessed via cosine similarity. If the sc/snRNA-seq data contains more cells than the spatial data (which is the typical case), a filter term in the loss function ensures that only the optimal subset of sc/snRNA-seq observations is selected. The output of Tangram is a probabilistic mapping, namely, a matrix denoting the probability of finding each cell from the sc/snRNA-seq data in each voxel of the spatial data. From this matrix, Applicants can obtain a deterministic mapping by assigning the most likely sc/snRNA-seq cell to each spatial voxel. Optionally, Applicants can also activate an entropy regularizer to ensure that the spatial probabilities of each cell are peaked over a narrow portion of space. Applicants did not need to use such feature, as all probabilities were peaked in practice in all cases analyzed in this study.) Tangram does not contain any hyperparameters, maps a hundred thousand cells in a few minutes (using a single P100 GPU), and is released as PyTorch module.

### Example 2—Tangram Maps Cells with MERFISH Measurements to Generate Genome-Scale High Resolution Expression Maps

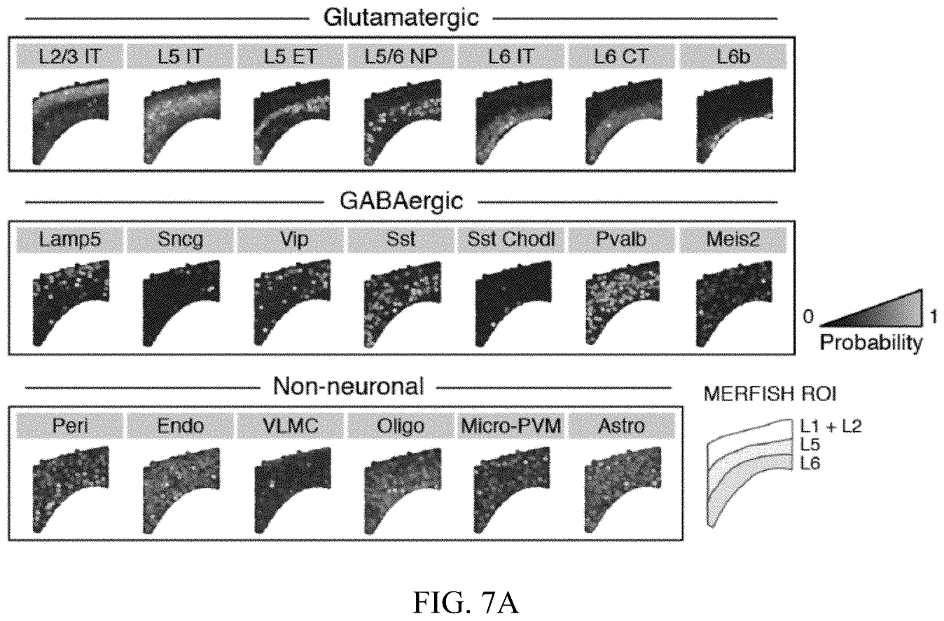

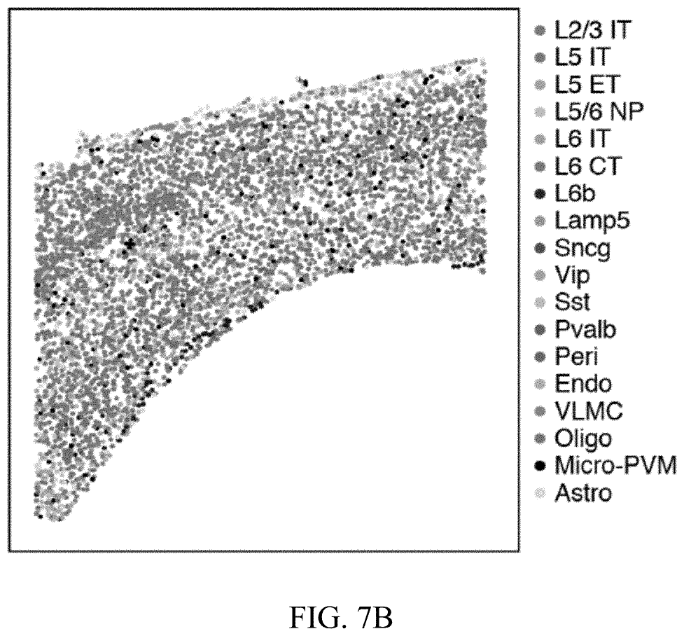

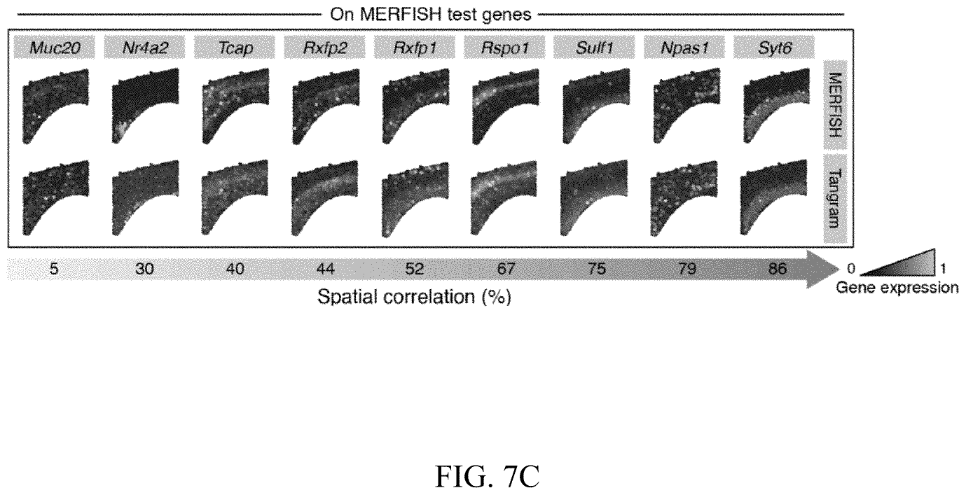

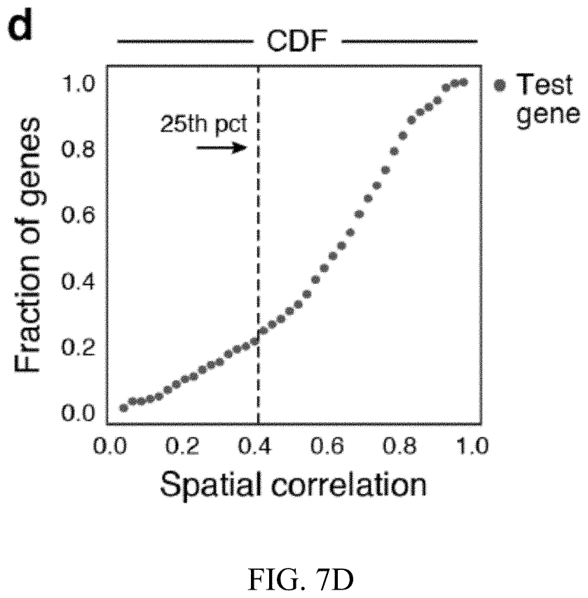

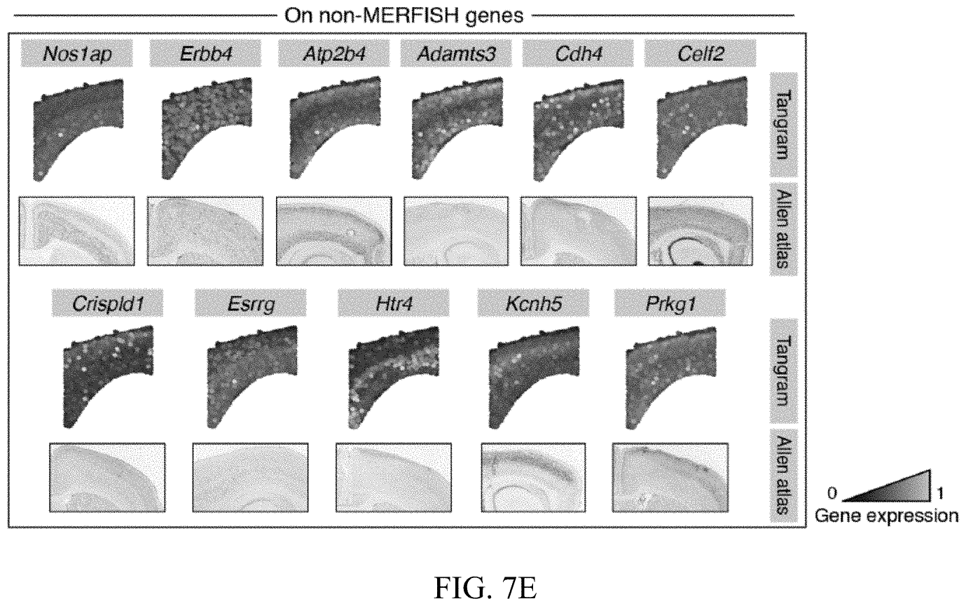

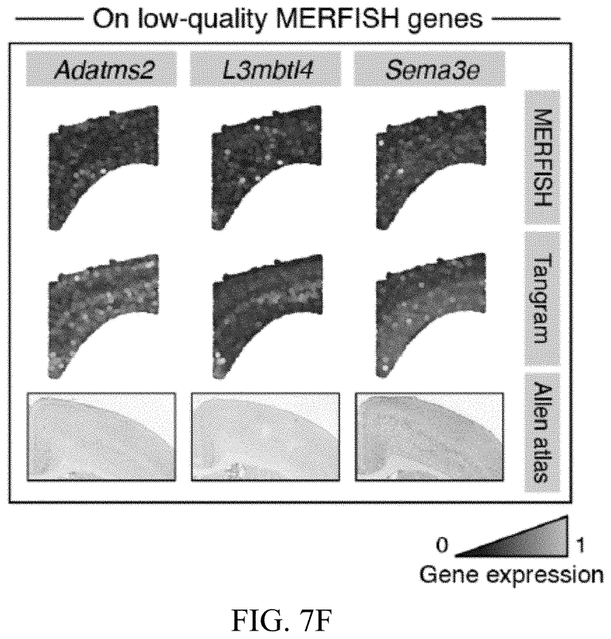

To apply Tangram, Applicants collected 160,000 snRNA-seq profiles using droplet-based RNA-seq (10Xv3, see for example³²), as part of the BRAIN Initiative Cell Census Network (BICCN), from the primary motor area (MOp) of health adult mouse brain. Each profile contains the expression of ˜27,000 genes, and was annotated following the recently delineated cell type taxonomy of neocortical areas³³, to 22 subclasses (hereafter, “cell types”)³⁴. Applicants mapped these snRNA-seq data with a high resolution MERFISH dataset of 254 genes, on a section segmented to 4,234 cells (FIG. 7). Applicants trained Tangram using 253 MERFISH genes (all genes but one were detected in the snRNA-seq data). 50% of the aligned profiles were neuronal, with a 6:1 ratio between glutamatergic and GABAergic cells, in accordance with their ratios in snRNA-seq.

To reveal the spatial distribution of cell types, Applicants combined the learned probabilistic mapping with the cell type annotations in the snRNA-seq data, and obtained the spatial probability distribution for each cell type (FIG. 7a). Glutamatergic cells show distinct cortical layer patterns of neuronal subpopulations whereas most, but not all, non-neuronal cells and GABAergic neurons are granularly distributed, as expected. Exceptions include non-neuronal VLMC cells (strongly localized in the first layer) and GABAergic Vip and Lamp5 cells, which appear more concentrated toward the upper layers. To verify that these distributions were not an artifact of the probabilistic approach, Applicants also visualized the cell type assignment from the deterministic mapping (i.e. only the most-likely cell is assigned to each spatial location) and observed similar patterns (FIG. 7b).

The learned Tangram model could predict spatial expression patterns well, as Applicants demonstrated by a leave-one-out analysis. Specifically, Applicants partitioned the genes into 252 training genes and a single test gene (unseen in the learning of the model). Applicants repeated the training 253 times, each time leaving out a different gene, to obtain prediction for each gene. As an evaluation score, Applicants computed the spatial correlation between each gene's real measurement and the predicted spatial pattern of that gene by the learned model. Overall, 75% of the 253 MERFISH genes are predicted with correlation >40% (FIG. 7d). To interpret these spatial correlations, Applicants chose nine genes with varied scores and visually compared the predicted spatial patterns to the MERFISH measurements (FIG. 7c). Importantly, the spatial patterns had good qualitative agreement for a broad range of spatial correlation values. For example, the prediction for Tcap (40% correlation) is in good accordance with its measurement. This is because when spatial resolution is at the single-cell level, correlation is highly sensitive to noise in gene expression or its measurement, such that somewhat lower correlation does not imply qualitative disagreement. This phenomenon is especially evident in highly sparse genes (e.g., Muc20): the sparse pattern is recapitulated, but the specific single cell locations are not precise, which may reflect the true nature of these patterns.

Mapping snRNA-seq data on MERFISH increases gene throughput to 27,000 genes, which Applicants validated for 11 selected genes with available ISH data in the Allen ISH dataset (FIG. 7e). Some genes exhibit strong, localized, patterns in striking similarity to those in the Allen images (Kcnh5, Nos1ap, Erbb4, Atp2b4, Celf2, Crispld1). For other genes the signal in the Allen ISH image is very dim compared to the predictions (Esrrg, Cdh4, Adamts3, Htr4, Prkg1), but a close inspection reveals agreement as well. This suggests that Tangram can reveal, spatial patterns for lowly-expressed genes, as Applicants will further demonstrate below (with Spatial Transcriptomics Visium data). Interestingly, Applicants obtained similar results when Applicants predicted withheld genes that were measured by MERFISH but had relatively lower-quality, possibly because of less optimal oligonucleotide probes used for these genes: the model predictions were consistent with ISH data, suggesting that the model can “correct” lower-quality signal (FIG. 7f). Correction of lower-quality measurements can also partly account for lower correlation scores between predictions and measurements (FIGS. 7c,f).

### Example 3—Accurate Correction of Low-Quality Transcripts in STARMap

To further investigate Tangram's correction of low-quality transcripts, Applicants analyzed a STARmap dataset². STARmap is a targeted in-situ technology which has been used to measured ˜1,000 genes in 3D resolution; however, increasing the number of measured genes trades-off with gene expression accuracy. In this dataset², 1020 genes are measured in 972 cells in a mouse brain slice from the visual area (VISp). Applicants map 11,759 SMART-Seq2³⁵ snRNA-seq profiles from the VISp area using 995 training genes present in both STARmap and snRNA-seq data.

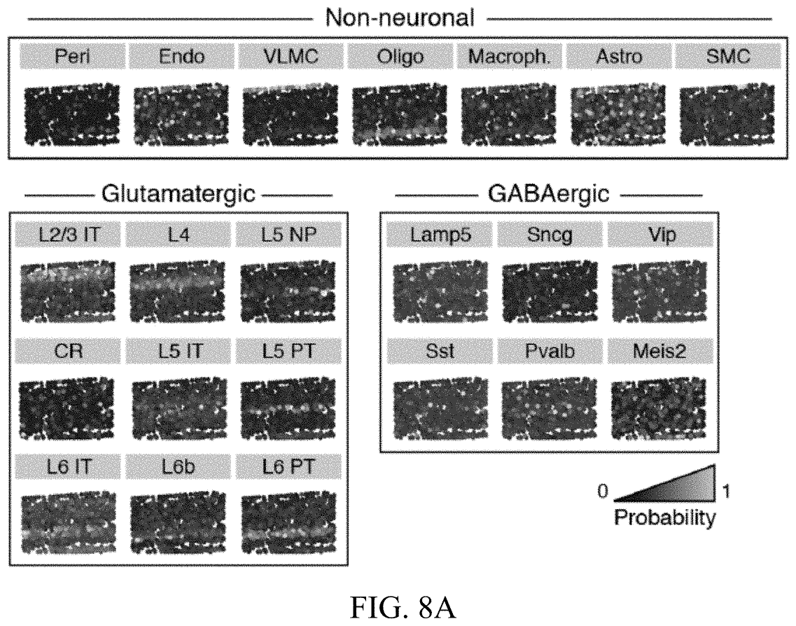

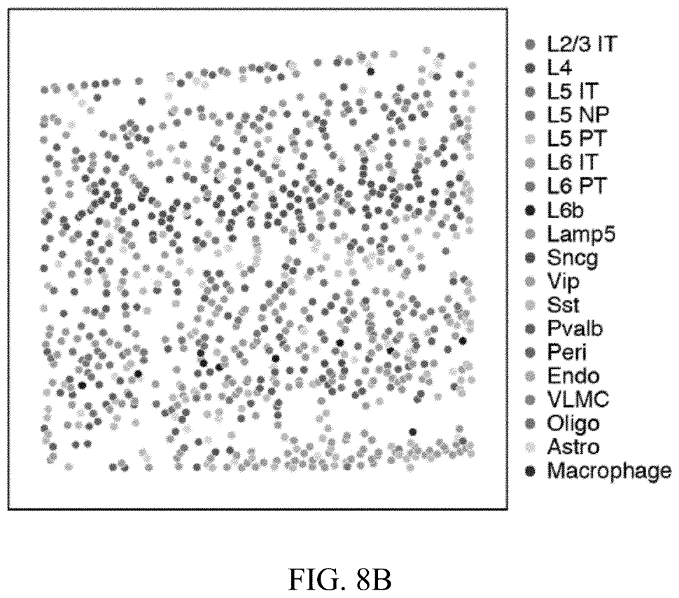

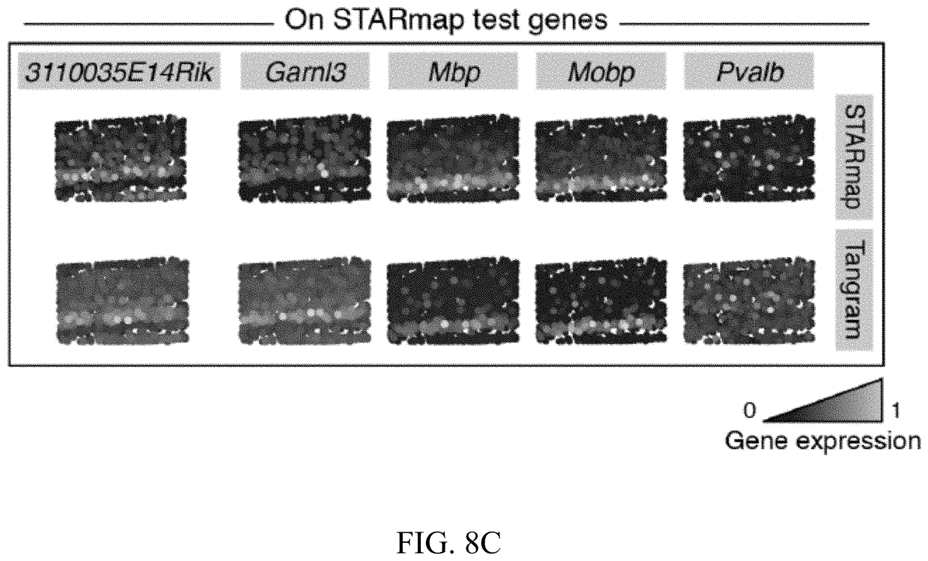

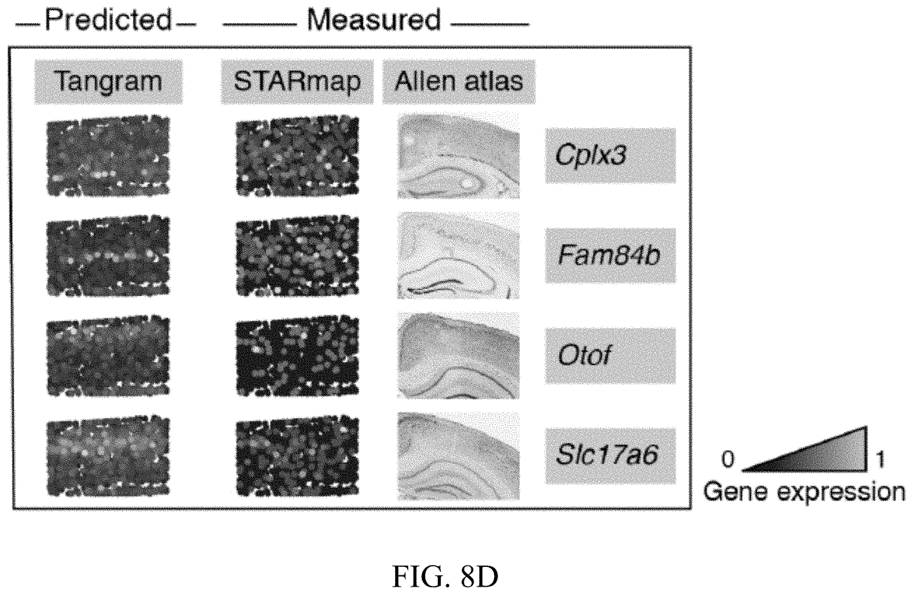

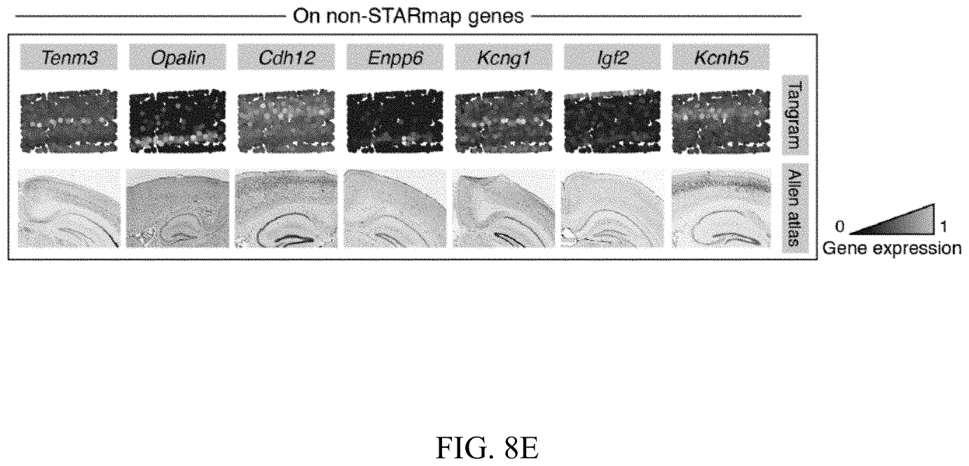

Inspecting cell type distributions from either probabilistic (FIG. 8a) or deterministic (FIG. 8b) mapping, Applicants confirmed that cell type patterns are consistent with those obtained with MERFISH from the motor area (FIGS. 7a,b). Despite minor cell-type annotation difference between the VISp and MOp snRNA-seq datasets, the model provides robust mapping. For example, while only the VISp (but not MOp) snRNA-seq dataset has an annotated glutamatergic L4 (layer four) cell subset, the model successfully revealed L4 in the MOp data (FIG. 8a) from predicting its marker genes (e.g. Kcnh5 in FIG. 8e and FIG. 7e). Finally, the STARmap dataset also contains sub-cortical tissue (defined as cells below the L6b layer), which allows us to further validate predictions by observing an expected subcortical concentration of oligodendrocytes (FIG. 8a).

Remarkably, Tangram not only predict expression for genes that were not measured by STARMap, but effectively corrects the spatial expression of low-quality genes (FIGS. 8c,d,e), as compared to Allen Brain Atlas ISH. First, when holding out each individual STARmap gene from the training, the predicted expression was typically consistent with direct measurements (FIG. 8c). Interestingly, for some genes the predicted localized patterns were not observed in measurements, especially for lower-quality genes (FIG. 8d). Remarkably, in these cases, the predicted pattern agreed well with images from the Allen Brain ISH Atlas (FIG. 8d), confirming the accuracy of the predictions, and Tangram's ability to correct gene expression of low-quality data. Finally, Tangram correctly predicted the expression of genes that were not measured by STARMap, including markers of cortical layers (Tenm3, Cdh12, Kcng1, Igf2) or sub-cortical tissue (Opalin and Enpp6) as assessed by their consistency with the Allen Brain ISH Atlas (FIG. 8e).

### Example 4—Single Cell Deconvolution and Histological Data Incorporation with Spatial Transcriptomics

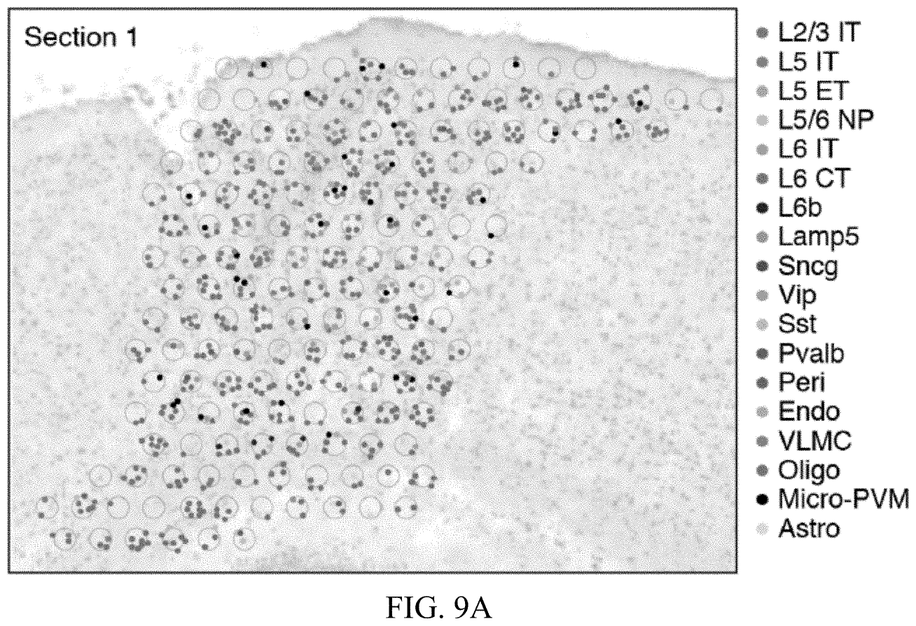

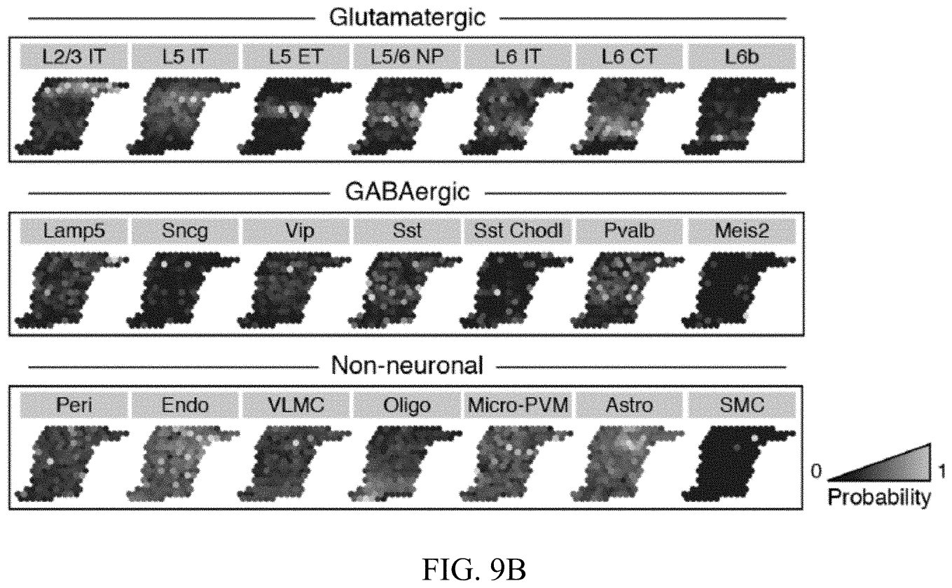

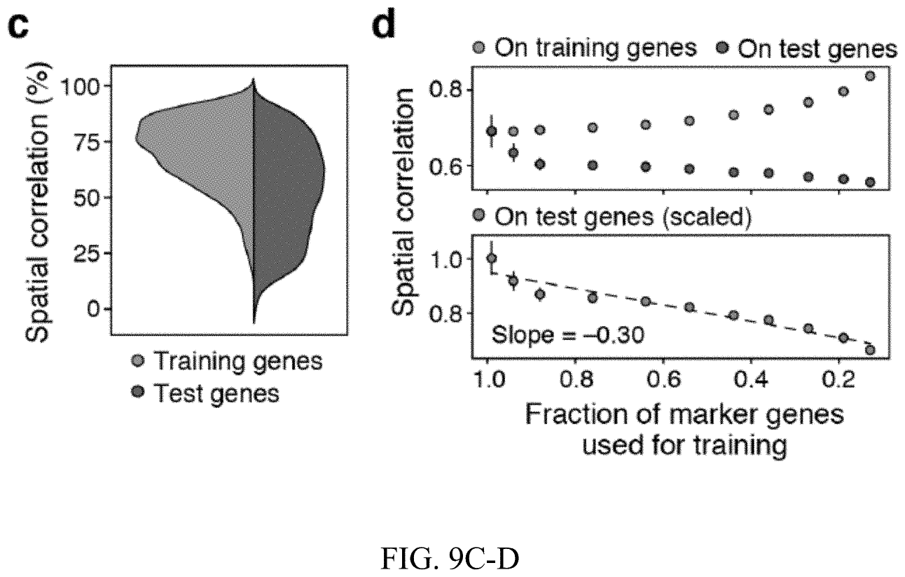

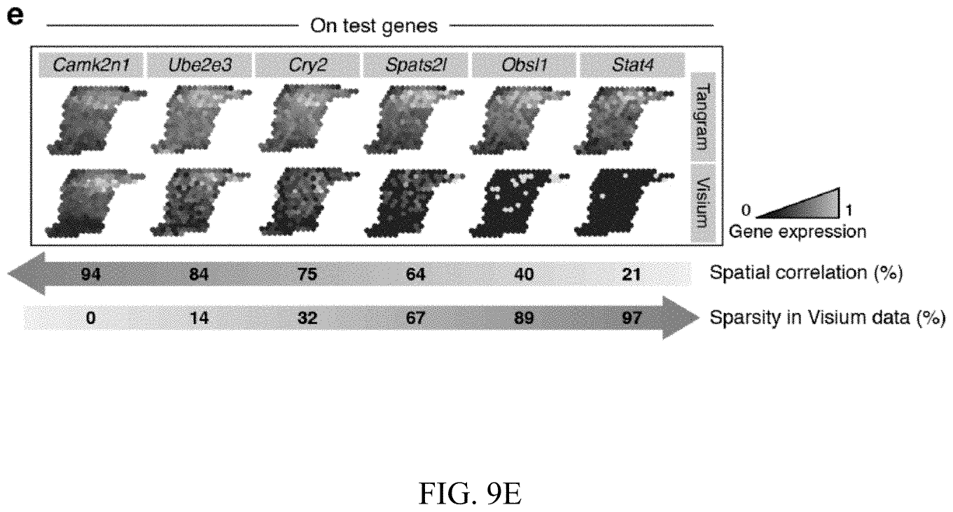

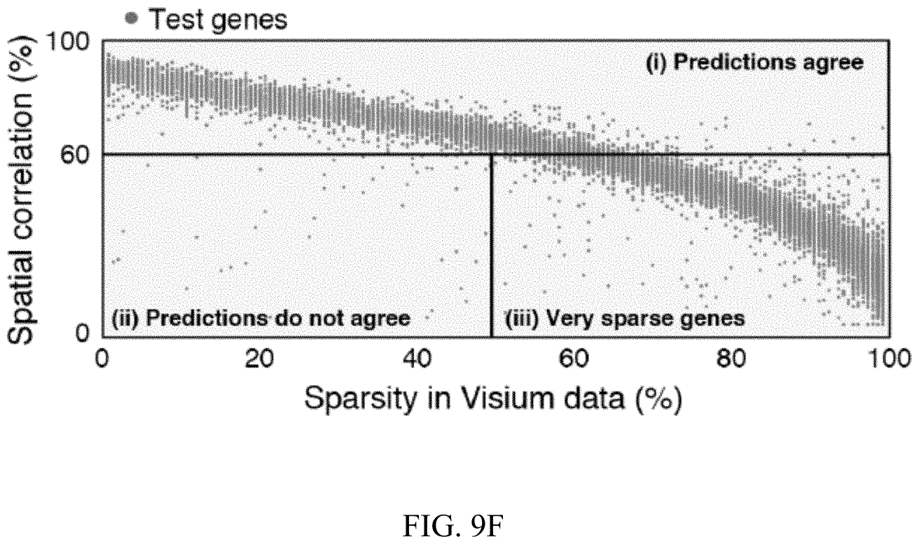

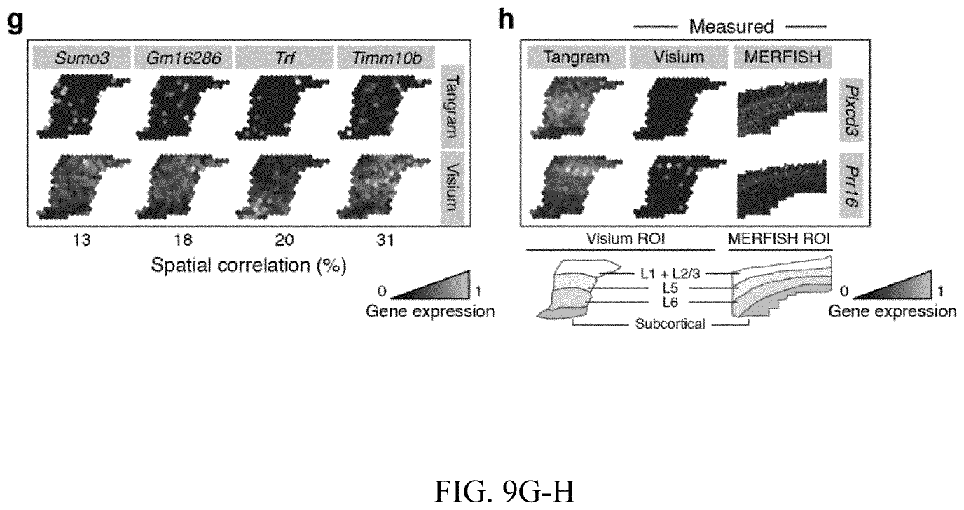

Next, Applicants focused on the deconvolution challenge in the context of lower-resolution Spatial Transcriptomics (Visium) data measuring 31,053 genes within 50 μm diameter circular spots in three coronal mouse brain slices (FIG. 9). This is followed by an H&E stain of the slice (section 1), with ˜160 circular spots on a region of interest. As single cells are visible in the stained images, Applicants segmented cells to directly estimate cell number within each spot, and overall counted 939 cells.

In Spatial Transcriptomics, unlike MERFISH and STARMap, Applicants possess a histological image, which identifies a specific depth in the brain coronal sectioning (more precisely, an anterior-to-posterior coordinate). This opens up two strategies for mapping: (1) map the full MOp snRNA-seq data, constraining the cell number to 939 (as for MERFISH and STARmap); this is more suitable for deconvolution, given the direct assignment on segmented cells; alternatively (2) Applicants can leverage the known anterior-to-posterior coordinate, and only map MOp snRNA-seq profiles collected at that depth (with a method Applicants describe later on); this should resolve smoother gene patterns, given the higher number of profiles as Applicants show in the following section.

For deconvolution, Applicants performed a deterministic mapping of each of the cells within each 50 μm voxel, to obtain cell type localization prediction at single-cell resolution (FIG. 9a). Applicants trained Tangram with a subset of the >30,000 genes, by selecting 1,237 training genes as the union of the top 100 marker genes of each cell type in the MOp snRNA-seq data (using a standard pipeline²⁰, Methods) that are detected in the Visium profiles. Unlike probabilistic mapping, Applicants assigned a discrete number of cells to each voxel (matching the number of segmented cells). Notably, rare cell types might not be assigned by deterministic mapping (e.g. Meis GABAergic neurons).

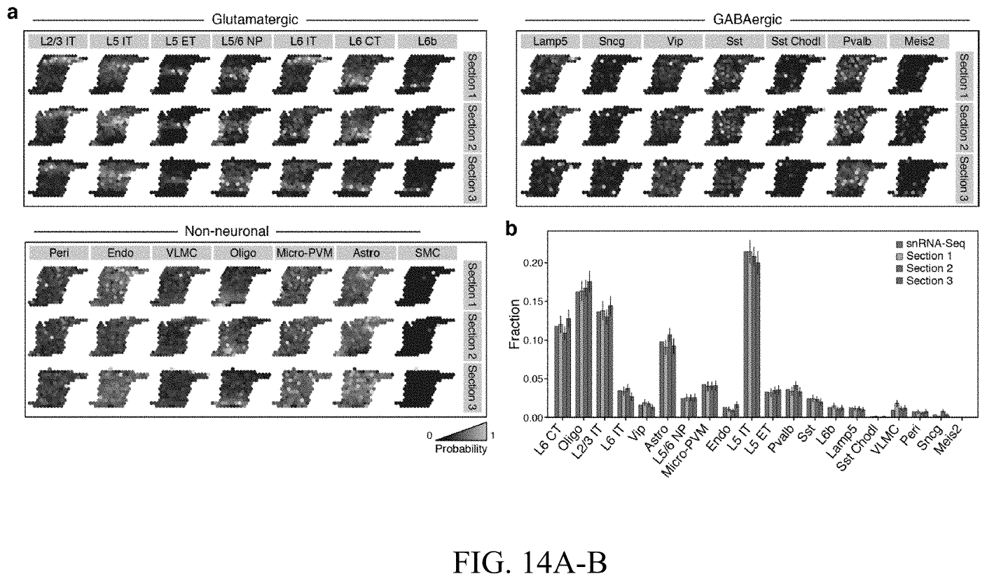

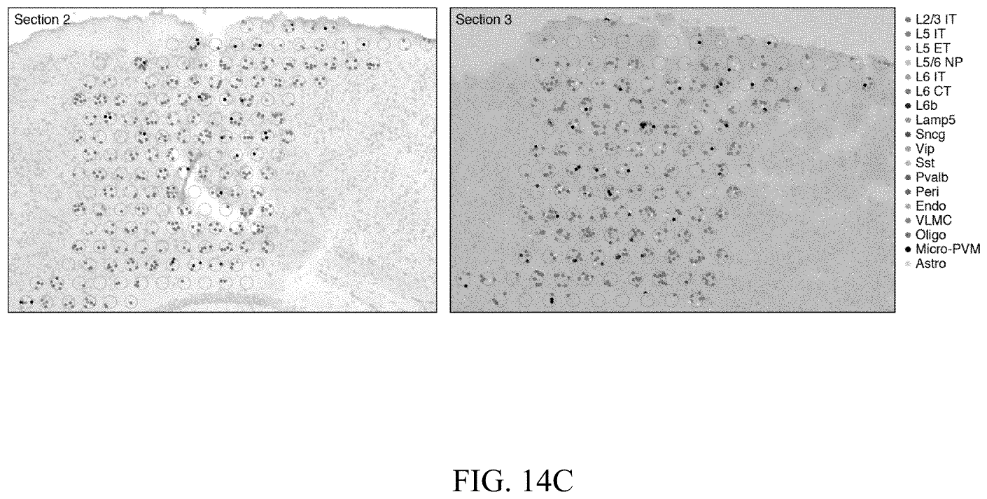

The deterministic mapping was successful, as demonstrated by comparing the mapped cell type ratios and those from the snRNA-seq data, which were consistent within Poissonian error (FIG. 14b). The mappings were also robust, as demonstrated by analyzing two other Visium datasets: a coronal section (section 2) consecutive to section 1 from the same specimen, and a coronal section collected at approximately the same posterior, provided publicly³⁶ (section 3) (FIG. 14c). Applicants note that assignment within a voxel is random: e.g., the model may predict that one microglia cell is contained in a certain voxel but not which cell it is.

### Example 5—Tangram Imputation of Dropouts in Spatial Transcriptomics

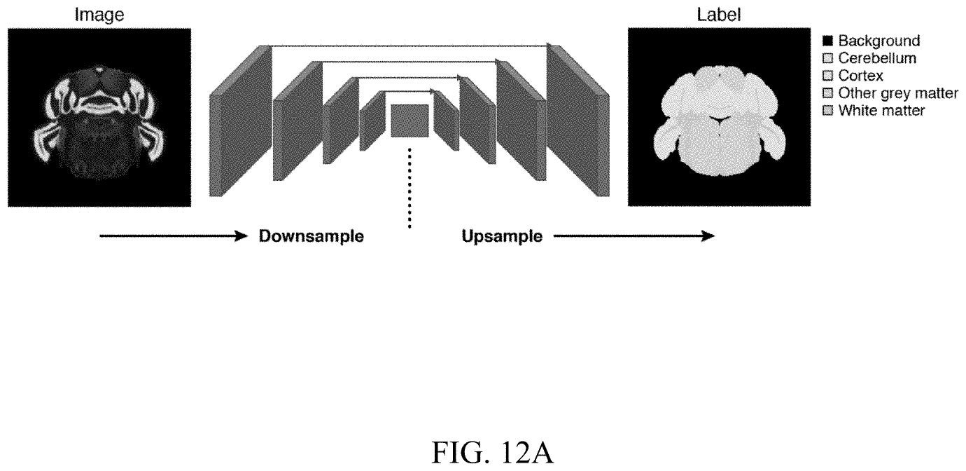

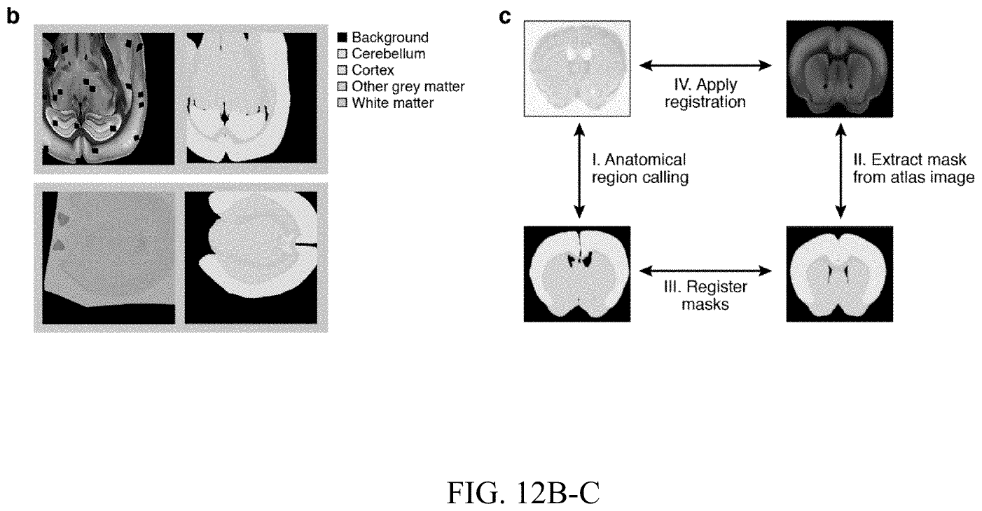

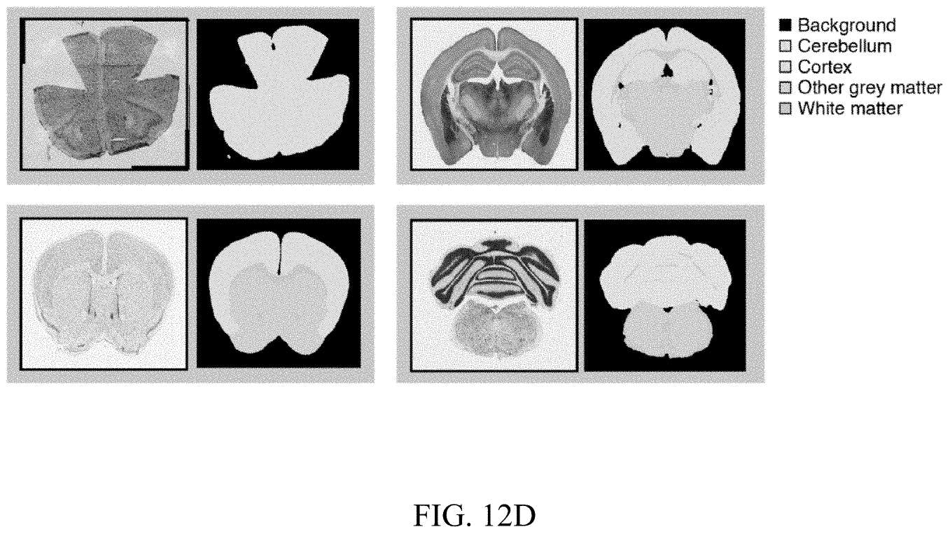

Next, leveraging the known anterior-to-posterior coordinates, Applicants mapped only MOp snRNA-seq profiles collected at that depth to impute gene expression and correct low-quality data (mostly due to dropouts). Specifically, during collection the MOp snRNA-seq data, Applicants tracked the dissected region, which Applicants annotated as “anterior”, “mid” or “posterior” based on its own histological data, as Applicants describe below (FIG. 12a, Methods).

As all three Visium histological images were called as “posterior” MOp, Applicants only mapped the 58,002 snRNA-seq profiles from “posterior” samples, using probabilistic mapping.

Tangram's mapping yielded higher resolution, finely localized, cell types (FIG. 9b, FIG. 14a). This included correct localization of L6b glutamatergic neurons, more concentrated presence of Vip⁺ and Lamp5⁺ GABAergic neurons in upper layers, positioning of Sst and Pvalb GABAergic neurons in deeper layers, and of Meis2⁺ and Sst⁺Chodl⁺ GABAergic neurons in rare sparse cell types. In a few cases, there is variation in the mapping between independent experiments, which is consistent with biological variation, for example, co-localization of cell types (e.g. Sncg and Vip GABAergic neurons) is detected across slices from the same batch (section 1 and section 2) but not in section 3; L6 IT cells are more localized in layer six in slice 3, and Vip neurons are more uniformly distributed in section 3 than in section 1 and section 2. Both findings are consistent with the expectations.

Importantly, Tangram correctly predicted spatial expression patterns from the mapped cells, when Applicants withheld those genes in the training and then compared them to the Visium measurements (FIG. 9c-f). Specifically, Applicants partitioned the genes into 1,237 training genes and 29,816 test genes unseen in the learning of the model, and used spatial correlation as before (FIG. 9c). The 90^{th} quantile of spatial correlation coefficients of training genes is >62%, setting a prediction threshold for test genes (FIGS. 9c,d): 50% of the testing genes exceed this threshold (FIGS. 9c,d). As the number of training genes in reduced from 1,237 to 123, so does the relative prediction accuracy (FIG. 9d), although it remains substantial. Inspection of spatial patterns from select test genes shows that while the predictions always result in a localized pattern in the upper layer, agreement against Visium measurements deteriorates as the gene is more sparsely detected in the original Visium experiment (FIG. 9e, where sparsity is defined as the fraction of voxels in which the gene is undetected).

Applicants hypothesized that this poor agreement could be due to technical “drop-outs” (˜15,000 test genes are entirely undetected in the Visium datasets). Supporting this hypothesis, there is a strong correlation between the prediction scores and data sparsity (FIG. 9f): 98% of non-sparse genes (sparsity<50%) are correctly predicted by the model (spatial correlation >62% threshold, FIG. 9f, region i), with only ˜70 non-sparse genes that are not well predicted (FIG. 9f, region ii). Non-sparse test genes that are not well predicted have predicted patterns that are sparser than Visium measurements, suggesting that the disagreement might be due to drop-outs in the snRNA-seq data (FIG. 9g). Finally, ˜80% of the transcriptome measured in Visium is highly sparse (FIG. 9f, region iii; the same genes are also too low to be detected by the Allen ISH atlas). This raises the possibility that the predictions may provide more accurate estimates of the real spatial expression for such genes. Supporting this, Applicants compared the predictions to measurement for the two genes available in both MERFISH and the sparse genes. In both cases, the predicted spatial patterns agree with MERFISH measurements (FIG. 9h). Future studies with additional genes could help with further validations.

### Example 6—Spatial Localization of Chromatin Accessibility Patterns with SHARE-Seq

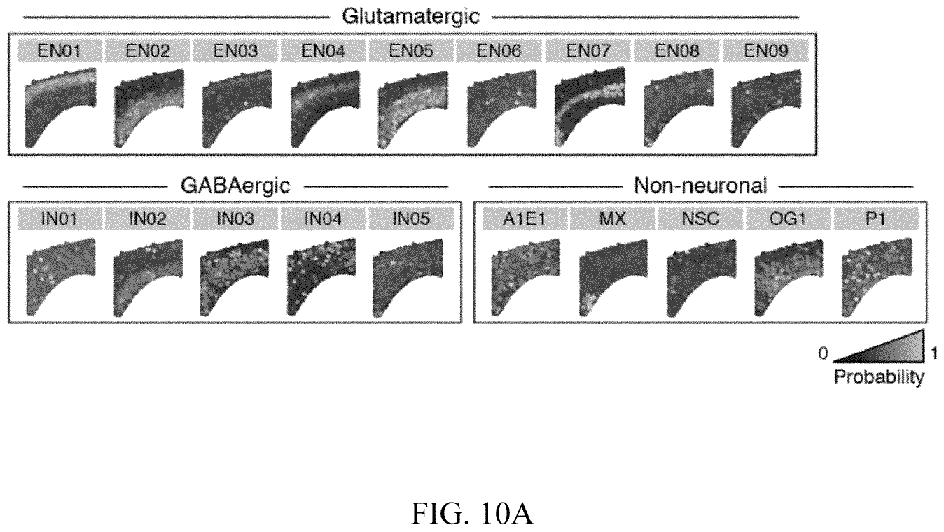

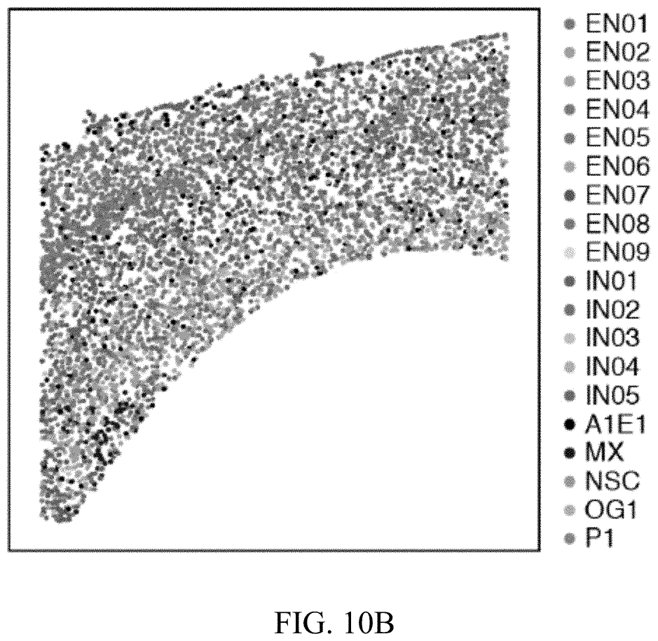

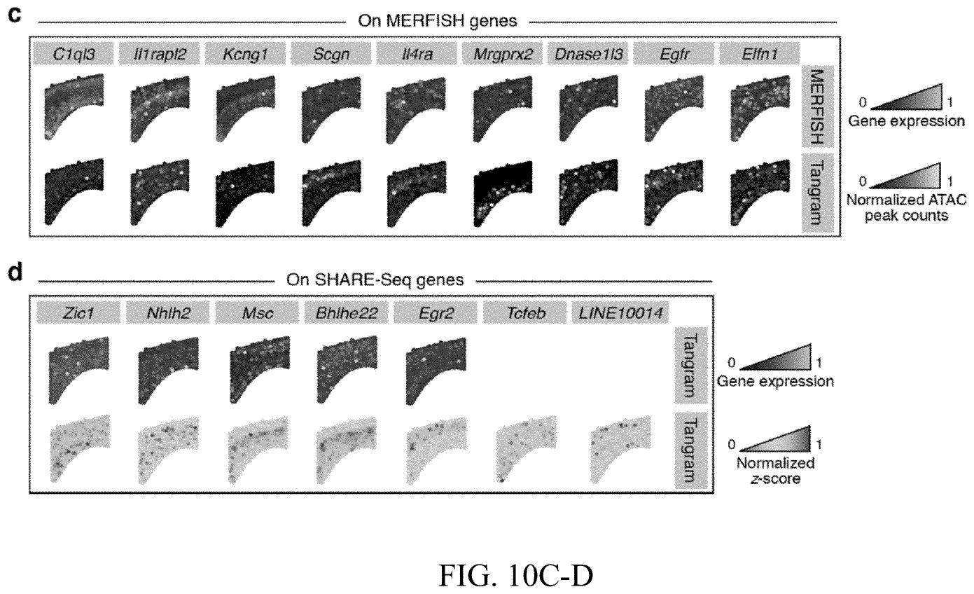

Applicants next used Tangram's successful spatial mapping through RNA, as a scaffold to map additional molecular profiles with no available spatial data, but that can be measured by single cell multi-omics. In particular, Applicants set to map a joint single cell RNA and ATAC-Seq, which Applicants profiled simultaneously in the >3000 cells from whole mouse brain by SHARE-Seg⁵ (detecting ˜18,000 genes) and annotated as nine glutamatergic cell subsets (EN, excitatory neurons), five GABAergic cell subsets (IN, inhibitory neurons) and five non-neuronal subsets (A1.E1, MX, NSC, OG1, P1)⁵ (no immune cells were captured, and cortical layer subsets were not annotated). Applicants aligned the SHARE-seq data to the MERFISH data using the snRNA-seq profiles, then transferred the snATAC-seq profiles of the cells to space, to visualize inferred spatial patterns of chromatin accessibility and transcription factor motif scores at single cells resolution (FIG. 10).

Applicants mapped SHARE-seq data both probabilistically (FIG. 10a) and deterministically (FIG. 10b) and obtained cell type distributions. The mapping reveals that EN01 are localized in layer L2/3, EN04 in layer 4, EN07 in layer 5/6, EN05 in layers 5 and 6 and EN02 in layer 6. Interestingly, IN02 seems more prominent in layer 6. Also, the non-neuronal cell type MX (labeled “Unconfirmed”⁵) appears concentrated to the bottom left part of the ROI, which does not resemble known patterns of cortical cell types. While the mapping is overall consistent, it is less biologically precise than in the previous cases, likely due to the lack of immune cells (missing “puzzle pieces” for Tangram) and the fact that the cells were not profiled specifically from the cortex.

Applicants used the snRNA-based mapping to infer spatial patterns of chromatin accessibility and transcription factor activity (FIGS. 10c,d), and compared them to spatial expression patterns. In some cases, gene expression is higher at a particular cortical layer but localization is not observed in the projected snATAC-seq (Clql3, Illrapl2, Kcng1). In other cases, the projected snATAC-seq forms spatial patterns, even though the corresponding predicted gene does not show a spatial pattern (Scgn, Il4ra, Mrgprx2). In only a minority of cases, Applicants observed correlation between snRNA-seq and snATAC-seq patterning (Dnasell3, Egfr and Elfn1). Applicants similarly visualized inferred spatial patterns of transcription factor motif activity scores (identified from the snATAC-Seq profile in each cell³⁷) (FIG. 10d). Interestingly, some of them exhibit a slightly localized pattern (Msc, Bhlhe22, Egr2), even for transcription factors whose predicted RNA was neither measured in MERFISH nor in SHARE-seq (e.g. Tcfeb and LINE10014).

### Example 7—Integration of Histological and Anatomical Atlas Support

In the mouse brain, there are already rich histological and anatomical atlases, with careful annotations and associated data, including in situ hybridization of individual markers. In those cases when histological data is directly available for specimens profiled in single cell atlases, those can serve as a bridge to these pre-existing atlases. This then both helps create a full atlas, in the context of a Common Coordinate Framework (CCF) for the brain, as well as provide spatial molecular data from legacy ISH.

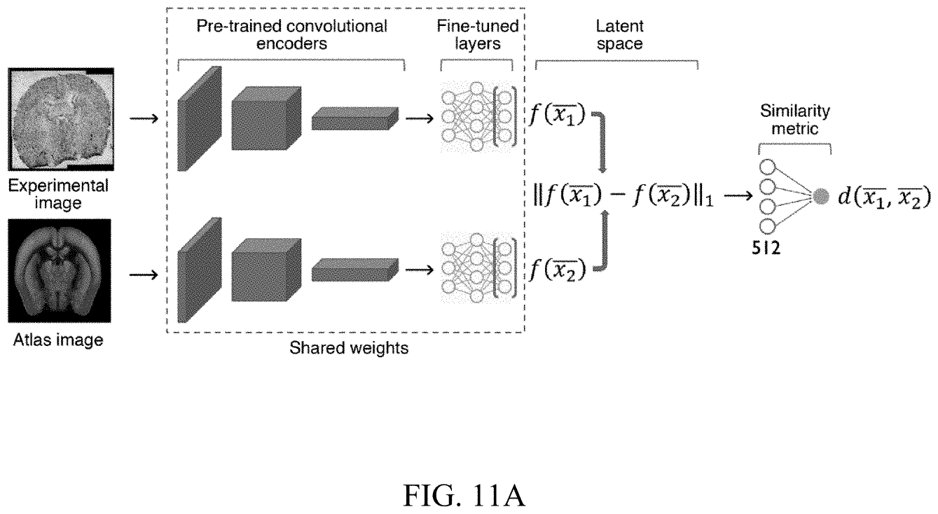

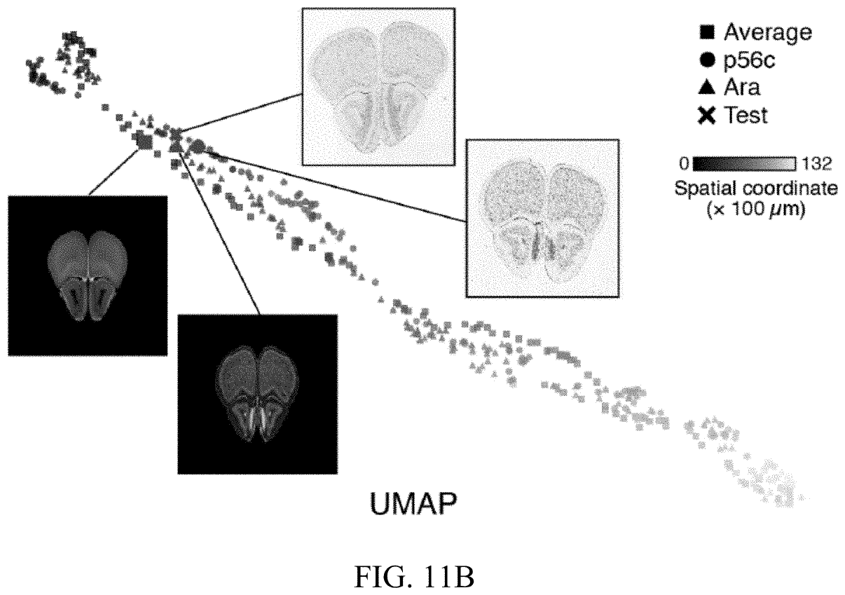

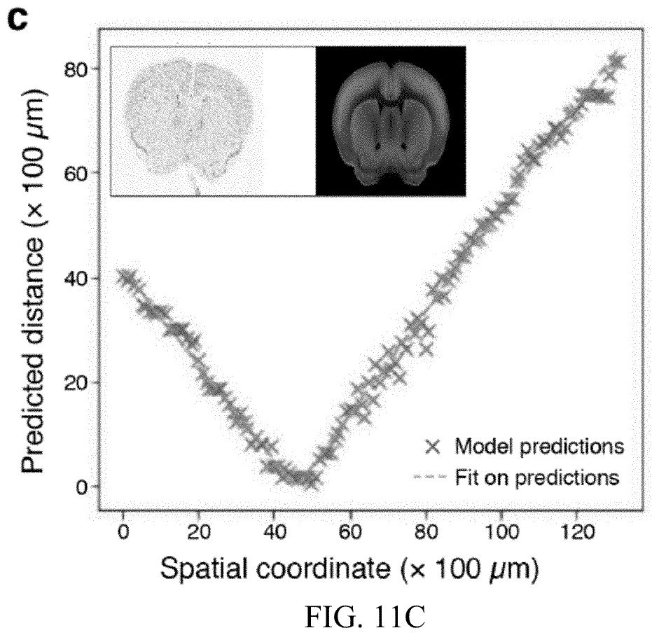

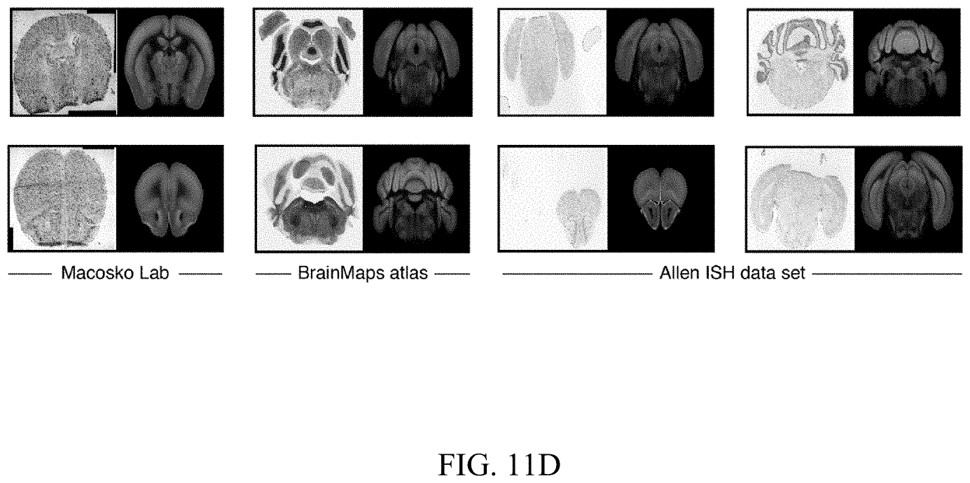

To this end, Applicants developed an additional module in Tangram to connect across scales by registering histology/spatial data on an anatomically annotated CCF, such as the Allen CCF for the adult mouse brain²⁸. As an alternative to methods that either require supervision or intact tissue, Applicants combine a Siamese neural network model with a semantic segmentation algorithm to produce full segmentation masks of anatomical images (FIGS. 11 and 12). The Siamese network model builds a latent space which allows a uniform encoding irrespective of technical artifacts in the images, such as the presence of “holes” in dissected regions (from which cells or nuclei were collected). The semantic segmentation model produces a segmentation mask with a color scheme that is compatible with the Allen ontology. Because Applicants produce a mask with matching colors, Applicants can then register the images automatically as Applicants do not need to provide corresponding landmarks; instead the anatomical regions in the mask are the landmarks.

Building on methods for face recognition¹⁶, Applicants learned a latent space using a Siamese network model' trained on mouse brain images (FIG. 11a, Methods). Applicants trained the model so that each image was encoded according to salient anatomical landmarks, whereas technical properties such as illumination or staining were factored out (Methods). The learned latent space displayed a one-dimensional manifold structure, where the “head” of the manifold contains images from the olfactory bulb, and the “tail”, images from cerebellum (FIG. 11b). The model predicted the image from the Allen CCF at the same coronal depth of the histological image. Applicants validated the predictions by checking consistency across the whole training set (FIG. 11c), and by visual inspection (FIG. 11d). Applicants then used the trained model to retrieve the image from the Allen CCF onto which Applicants register the histological image.

Next, Applicants segmented the images to generate a custom mask for the images using the same color scheme adopted by the Allen ontology. For this, Applicants applied semantic segmentation¹⁸, and segmented five classes in the histological image (FIG. 12a): background, cortex, cerebellum, white matter and other grey matter. As the training set is scarce, Applicants adopted a combination of transfer learning and heavy augmentation during training (FIG. 12b) and validated it by inspecting predictions on test atlases (FIG. 12d). Finally, Applicants combined segmentation with the Siamese model, to obtain a fully automated registration pipeline (FIG. 12c), leveraging the fact that registering two masks (one on the Allen image and one on the image of the sample) is a simpler problem than registering the two images directly.

### Example 8—A Learned Histological and Anatomical Atlas of Single Nuclei From the Somatomotor Mouse Cortex

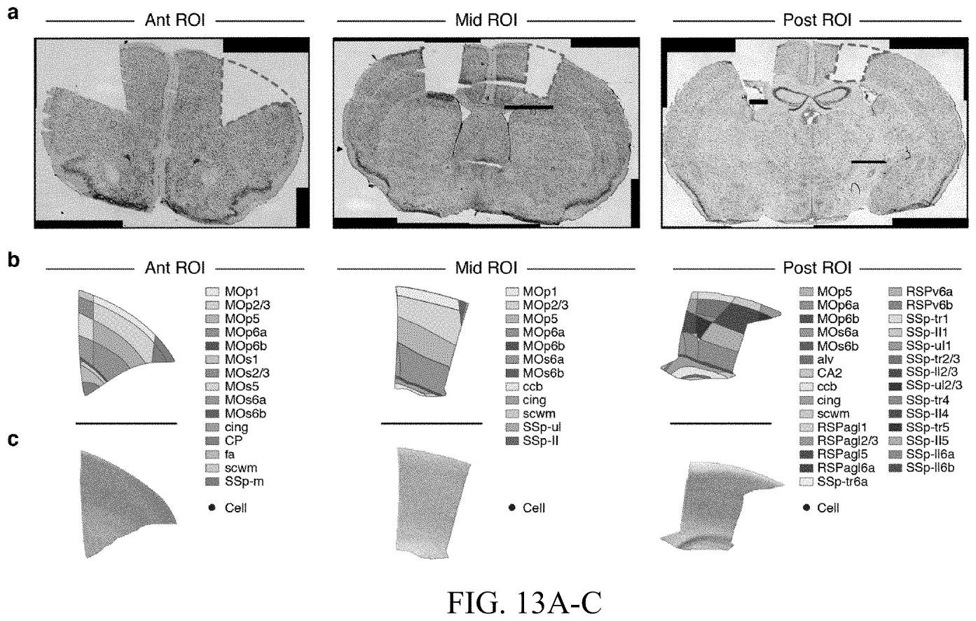

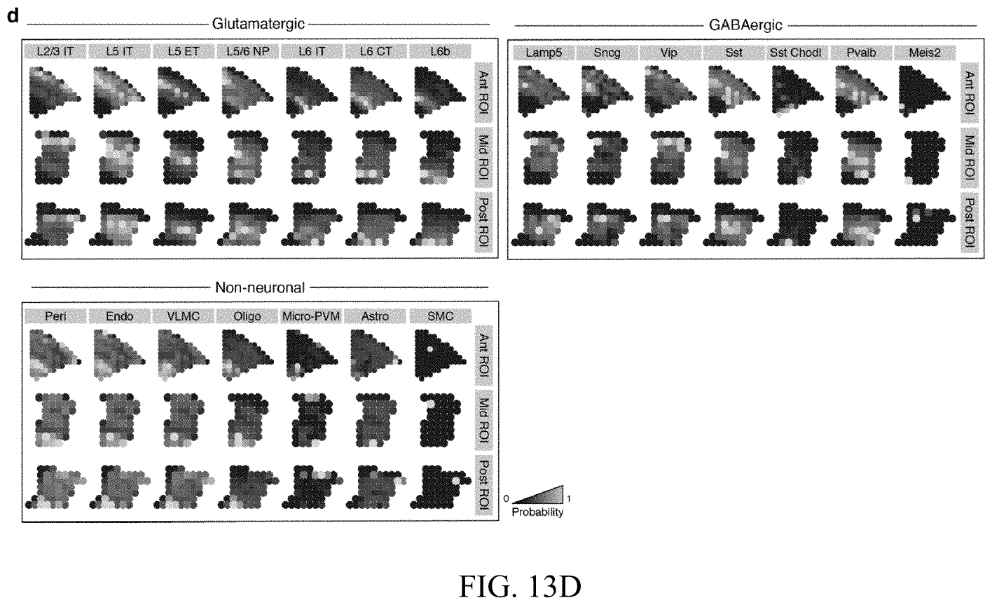

To demonstrate this final framework, Applicants assembled an integrated atlas of the somatomotor area of the healthy adult mouse brain using publicly available atlases. As noted above, Applicants collected ˜160,000 snRNA-seq profiles from three dissected regions of interest (ROIs) in the somatomotor area (FIG. 13a). Applicants used a lab procedure³⁸ where nuclei are isolated from a biopsy “punch” in a frozen dissected region. To help relate this region to the known anatomy, Applicants also obtained stained histological sections on the punched section, which are approximately 200 μm deep (Methods).

Applicants applied Tangram's anatomical mapping module to the histological images to precisely locate the region of dissection on the Allen CCF, and then queried the Allen Mouse Atlas to estimate spatial gene expression at 200 μm resolution and the Blue Brain Cell Atlas¹⁹ to compute the expected cell density in each spatial voxel (FIG. 13c). Tangram then computed an anatomical map from the Allen Reference Atlas, and used it post-mapping to estimate the anatomical region to which each cell has been mapped (FIG. 13a). Applicants repeated this procedure for the three ROIs, and finally mapped the snRNA-seq profiles to their corresponding ROIs. (Applicants used the same anatomical mapping module to select nuclei for mapping from a matching histological section to that measured by Spatial Transcriptomics; above.)

The mapping predictions for cell types across the three ROIs, were self-consistent albeit less accurate than mappings using the higher resolution spatial technologies (FIG. 13d). Cortical layers were successfully recovered across the three ROIs, but L5 IT and L5/6 NP display a lower level of localization than in the other cases. GABAergic neurons predictions are biologically sound and Applicants observed a more concentrated presence of Vip+ and Lamp5+ in upper layers, as observed with Visium-based mapping. Non-neuronal predictions did not recover sparse mPVM and overly concentrated Peri, Endo and VLMC cells in the subcortical part. Overall, the mapping results confirmed that glutamatergic cell type patterning is simpler to reconstruct than sparse, granular, cell type patterns typical of non-neuronal cell types, the latter requiring finer signals from modern spatial technologies.

### Example 9—Discussion

Gene expression in the brain and other organs exhibits a variety of spatially-organized patterns on different length scales from the microenvironment, to histology to anatomy, whose knowledge is central to unraveling biological function. Spatially resolved transcriptomic data provide an opportunity to reveal such patterns, but are currently limited by spatial resolution or the number of genes measured, and connecting them to other levels or organization can require substantial experimental efforts. Here, Applicants harmonized snRNA-seq data with in-situ, histological and anatomical data, to address these limitations, towards a high resolution, integrated atlas.

Here, Applicants developed and used Tangram to tackle various scenarios, all of which involved aligning snRNA-seq data onto a certain type of spatial data collected from the adult mouse brain cortex, from high resolution MERFISH and STARMap, through mid-resolution Spatial Transcriptomics, and to ISH associated with histological and anatomical coordinates. In each case, Applicants validated the computational alignments by recovering consistent spatial maps of cell types and predicting the expression of holdout genes.

Each type of spatial measurement modality benefits particularly from one aspect of Tangram: for high resolution targeted datasets (smFISH, MERFISH, STARmap), Tangram expands from signature to genome-wide patterns; for lower resolution Spatial Transcriptomics dataset (Visium), Tangram yields single-cell resolution; for datasets with higher gene throughput (STARmap and Visium), Tangram detects and corrects low-accuracy gene expression patterns; for multi-modal single cell data (SHARE-seq), Tangram uses one modality to generate spatial patterns for the other, thus generating spatial multi-modal maps. Finally, histology allows registration to the Allen CCF and integration between the cellular and the anatomical scale.

In the analyses, a few hundreds of marker genes, stratified across cell types, sufficed to map the mouse brain cortex transcriptome-wide, observing consistent cell type patterns in all cases. Notably, while cell mapping can rely on even fewer genes (i.e., smFISH, 22 genes measured, FIGS. 6b,d), Applicants could not successfully predict transcriptome-wide spatial gene expression in that case, in contrast to the success with MERFISH (254 genes measured). This suggests that at least a few hundred marker genes are required to comprehensively map the mouse brain cortex, at least in the context of cell types. As Applicants expand studies of the brain to other patterns—such as short-term responses that characterize more transient cell states and programs—the optimal number of marker genes required for mapping could depend not only on the number and diversity of cell types in the tissue and specificity of marker genes, but on the structure of other gene programs and their inter-relations. Tangram, using both genome-wide and targeted spatial scaffold data can help assess the extent of markers needed to capture a response.

Future applications of Tangram with more than a single spatial scaffold at a time (e.g., taking both MERFISH, Visium and snRNA-seq as inputs simultaneously) should help reconcile a complete integrated spatial atlas of gene expression in the brain. Moreover, Tangram could be applied in an iterative fashion across the modalities. For example, as Applicants showed Tangram can correct gene expression in spatial methods when the measurements of the specific genes have lower quality. Such output can then be used as a novel spatial dataset onto which to re-align the snRNA-seq profiles. This technique, called active learning, will be explored in future studies. Finally, as additional spatial transcriptomics technologies arise, Tangram can flexibly take them as input. For example, it could be applied to SLIDE-seq2 data³⁹, which has a spatial resolution of 10 μm (compared to Visium's 50-55 μm) and is more robust to technical dropouts³⁹.

An interesting case study of Tangram is its ability to resolve spatial patterns of multi-modal data, when only one modality is available in the spatial scaffold, as Applicants demonstrated using SHARE-seq data to predict spatial patterns for scATAC-seq data. This approach can be adopted with every technology that profiles the transcriptome at single cell level with an additional modality: examples include CITE-seq⁴⁰ (cellular proteins) and Patch-seq⁴¹ (electrophysiological and morphological properties of neurons). Alternatively, one can independently measure various modalities in different subpopulations at single-cell level, integrate them by learning a common latent space^{24,42,43}, and then input this integrated multi-modal data to Tangram to resolve multi-modal spatial patterns. Aligning multi-modal data is particularity intriguing in cases for which there is no assay for spatially resolving data of a certain modality. For example, chromatin accessibility could not be spatially resolved at single-cell level until very recently⁴⁴.

Finally, while the work focused on a specific region in the mouse brain, Tangram is applicable to any brain region, towards its complete atlas, and to any other organ, as well as disease tissue. To integrate across scales, Tangram's registration pipeline requires a CCF and is therefore currently applicable to a few organs. At present, the mouse brain possesses the most advanced and well-developed CCF²⁸, but efforts are underway to construct analogous reference maps for different organs⁹, towards the construction of cell atlases of all organ in mouse and human.

### Example 10—Materials and Methods

### Tangram Mapping Algorithm

Introduction. Applicants use the index i for cells (i.e. snRNA-seq data), k for genes and j for spatial voxels (circular spots, pucks, etc.). The goal is to learn a spatial alignment for the cells, organized as a matrix S with dimensions n_{cells}×n_{genes} where n_{cells} is the number of single cells, such that Sᵢₖ≥0 is the expression level of gene k in cell i. In order to map, Applicants voxelize the spatial volume at the finest possible resolution (which depends on the mapping case, e.g. 200 μm when mapping with the Allen Brain Atlas, individual cells when mapping with MERFISH, etc.), and index the voxels in an arbitrary one-dimensional fashion. Applicants then introduce two quantities: the nᵥₒₓₑₗₛ×n_{genes} gene expression matrix G, where Gⱼₖ≥0 denotes the expression of gene k in voxel j (Applicants do not assume that G and S measure gene expression using the same unit of measures), and a nᵥₒₓₑₗ-long vector \vec{d} of cell densities, where 0≤dⱼ≤1 is the cell density in voxel j, and Σⱼⁿ^{ᵥₒₓₑₗ} dⱼ=1.

Applicants aim to learn a mapping matrix M with dimension n_{cells}×nᵥₒₓₑₗₛ, such that Mᵢⱼ≥0 is the probability of cell i of being in voxel j. Therefore, Applicants require a probability constraint Σⱼⁿ^{ᵥₒₓₑₗ} Mᵢⱼ=1. The mapping strategy is probabilistic, perform a soft assignment. From the mapping matrix M, Applicants further define two quantities: M^{T}S, the spatial gene expression as predicted by the mapping matrix, and the vector \vec{m} with components mⱼ=Σᵢⁿ^{_{cells}} Mᵢⱼ/n_{cells} for the predicted cell density in voxel j. Finally, Applicants define the softmax function along the voxel axis for any given matrix \tilde{M} (with dimensions n_{cells}×nᵥₒₓₑₗₛ). The resulting matrix M has elements:

$${M}_{i j} = softmax {( \tilde{M} )}_{i j} = \frac{{e}^{{\tilde{M}}_{ij}}}{{\sum }_{l}^{{n}_{v o x e l s}} {e}^{{\tilde{M}}_{ι l}}} .$$

By applying the softmax, Applicants ensure that 0≤Mᵢⱼ≤1 and Σⱼⁿ^{ᵥₒₓₑₗ} Mᵢⱼ=1.

Mapping algorithm. To learn the mapping matrix, Applicants minimize the following objective function with respect to \tilde{M} (note that in the objective Applicants use M=softmax(\tilde{M})):

Φ(\tilde{M})=KL(\vec{m}, \vec{d})−Σₖⁿ^{_{genes}} cosₛᵢₘ((M^{S}S)_{*,k}, G_{*,k})−Σⱼⁿ^{ᵥₒₓₑₗₛ} cosₛᵢₘ((M^{T}S)_{j,*}, G_{j,*}), (1)

where KL indicates the Kullback-Leibler divergence and cosₛᵢₘ is the cosine similarity function. The first term is the density term: Applicants enforce that the learned density distribution is as similar as possible to the expected density. The second term is the gene/voxel expression term: it enforces that the predicted expression for each gene over the voxels is proportional to the expected gene expression over the voxels. The third term is the voxel/gene expression term: for each voxel, the predicted gene expression needs to be proportional to the expected gene expression. Minimization is obtained via gradient-based optimization using the PyTorch library.

Using the objective (1), Tangram maps all sc/snRNA-seq profiles onto space. If the number of sc/snRNA-seq profiles is higher than the known number of cells in the spatial data, Tangram can instead filter the sc/snRNA-seq profiles and learn the optimal subset of sc/snRNA-seq profiles that best explains the spatial data. The latter approach is explained next.

Mapping with a filter. Applicants introduce a filter vector \vec{f} of dimension n_{cells} so that cell i can either be mapped (fᵢ=1) or not mapped (fᵢ=0). To filter, Applicants multiply each row of the single-cell matrix, S_{i,*}, and each row of mapping matrix, M_{i,*}, by fᵢ, as shown below in the new objective. The filter values fᵢ are learned during training, in order to retain the cells that best explain the spatial data. To explicitly promote Boolean values (i.e. 0 s or 1 s) in the filter values, Applicants add a filter regularizer in the objective. To enforce the total number of filtered cells, Applicants introduce a count term: a soft constraint in the objective that promotes a number of mapped cells in the filter equal to n_{target_cells}. With this in mind, Applicants formally define the objective. Applicants define a real vector \tilde{\vec{f}} of dimension n_{cells} and define fᵢ=σ(\tilde{f}ᵢ), where Applicants apply the sigmoid function a to ensure that 0≤fᵢ≤1. Applicants then define S^{f}=diag(f)·S and M^{f}=diag(f)·M, namely, the filtered versions of the single cell matrix and the mapping matrix, respectively. Applicants also define \vec{m}_{f}, vector with components m^{f}ⱼ=Σᵢⁿ^{_{cells}} M^{f}/Σᵢⁿ^{_{cells}} fᵢ, as the predicted density of filtered cells in voxel j. The objective function, which Applicants minimize with respect to \tilde{M} and \tilde{\vec{f}}, is:

Φ(\tilde{M}, \tilde{\vec{f}})=KL(\vec{m}^{f}, \vec{d})−Σₖⁿ^{_{genes}} cosₛᵢₘ((M^{T}S^{f})_{*,k}, G_{*,k})−Σⱼⁿ^{ᵥₒₓₑₗₛ} cosₛᵢₙ((M^{T}S^{f})_{j,*}, G_{j,*})−λᵣ_{₁} Σ_{i,j}ⁿ^{_{cells,}} ⁿ^{ᵥₒₓₑₗₛ} Mᵢⱼ log(Mᵢⱼ)+abs(Σᵢⁿ^{_{cells}} fᵢ−n_{target_cells})+Σᵢⁿ^{_{cells}}(fᵢ−fᵢ²). (2)

The last two terms correspond to the count term and the filter regularizer, respectively.

Annotations transfer. The output of the mapping algorithm is the learned mapping matrix M (with, optionally, the learned filter \vec{f}). Once the mapping outcome is computed, any kind of annotation can be transferred from the sc/snRNA-seq data onto space.

Applicants define A as the annotation matrix with dimensions n_{cells}×nₐₙₙₒₜₐₜᵢₒₙₛ, where nₐₙₙₒₜₐₜᵢₒₙₛ is the number of different annotations characterizing single cells (e.g., genes, cell types, or any other modality). If annotations are categorical values (such as cell types), Applicants one-hot encode them over multiple columns in A. Annotations in space are retrieved by computing:

A_{transf}=M^{T}A,

or, if the filter has also been learned, via:

A_{transf}^{f}=M^{T}(diag(f)·A).

The computed matrix A_{transf} has dimensions nᵥₒₓₑₗ×nₐₙₙₒₜₐₜᵢₒₙₛ, and therefore denotes the annotations in space.

Cell type calling. When A describes cell types, A_{transf} describes the probabilistic counts for each cell type in each cell voxel. This corresponds to probabilistic mapping and can be interpreted as the mixture of cell types which best explain the in situ gene expression. When the technology allows for single-cell spatial resolution, voxels correspond to single cells in space. In this case, probabilistic mapping can be seen as an unnormalized probability distribution over cell types for the voxel/cell. As a consequence, for technologies with single-cell spatial resolution, Applicants can define a deterministic mapping as the mapping assigning the most likely cell type in this distribution to each spatial voxel/cell.

Mapping spatial data from targeted technologies. smFISH (FIG. 6), MERFISH (FIG. 7), and STARMap (FIG. 8) allow for single-cell spatial resolution, therefore, the number of spatial voxels needs to be equal to the number of cells. As snRNA-seq profiles are typically more numerous than MERFISH voxels, Applicants can use the mapping with the learned filter, namely, Eq. (2). In this case, n_{target_cells}=nᵥₒₓₑₗ and the expected density \vec{d} is uniform anu equal to

$${d}_{j} = \frac{1}{{n}_{v o x e l}}$$

for all j. This enforces that each cell is mapped to one voxel only and vice versa. If the number of available single cells is lower than the number of spatial spots, Applicants can instead map with Eq. (1), using the same constant density \vec{d}.

For the MERFISH case, Applicants mapped 58,022 10Xv3 snRNA-seq profiles in 4,951 spatial spots. From the 26,944 genes in the snRNA-seq data, Applicants selected 1,408 marker gene as the top 100 marker genes stratified across the 22 cell types. Applicants mapped using the intersection between these marker genes and the 254 MERFISH genes, which corresponded to 253 genes.

For the smFISH case, Applicants mapped 11,759 SMART-Seq2 snRNA-seq profiles in 4,840 spatial spots. In this case, 40,056 transcripts are measured in the snRNA-seq data. Only 22 genes were measured in smFISH, all of which were also present in the snRNA-seq data. Therefore, Applicants used all 22 genes for mapping.

For the STARMap case, Applicants used the same snRNA-seq data as for smFISH, which Applicants mapped on 1,550 spatial spots. Applicants took the intersection of 995 genes between the 1,020 STARmap transcripts and the 40,056 transcripts in the snRNA-seq data. Applicants used these 995 genes for mapping.

The algorithm converges after 1,200 epochs in all the experiments.

Mapping Visium data. Applicants began by identifying the Post ROI on the Visium histological image using the registration pipeline (below). Next, Applicants segmented the cells within the ROI using the software ilastik (www.ilastik.org). Applicants then built the density vector \vec{d}, by computing the cell density inside each voxel (i.e., Visium circle, as in FIG. 6e). Applicants mapped using the objective described in Eq. (1). Mapping yields the matrix M, which Applicants used to generate probability maps for the cell types within the ROI. To deconvolve, Applicants mapped using Eq. (2), to constrain the expected number of cells in each Visium voxel. Specifically, Applicants used n_{target_cells}=n_{seg}, where n_{seg} is the total number of segmented cells in the Visium ROI, to enforce that only a subset of cells is actually mapped. The count term combined with the density term led to the expected number of mapped cells in each Visium voxel. After training, Applicants assigned the types of the cells mapped within each voxel randomly to specific segmented cells within that voxel.

For probabilistic mapping on Visium data, Applicants ran the optimizer for 300 epochs to reach convergence. At the end, more than 99% cells were assigned to an individual voxel with probability greater than 50%. For used deterministic mapping in deconvolution, Applicants trained the optimizer for 6,000 epochs to reach convergence. At the end, more than 99% cells were assigned to an individual voxel with probability greater than 50%. For section 1 dataset, the number of cells filtered (fᵢ>0.5) is 880 (89% of segmented cells). Segmented cells for which there is no filtered mapped cell are not shown in the figures.

For both probabilistic and deterministic mapping, Applicants used 58,022 10Xv3 snRNA-seq profiles for 162, 161 and 134 spatial spots, respectively, in section 1, section 2 and section 3. Among the 26,944 transcripts in the snRNA-seq data, 1,408 marker genes were selected. Applicants mapped using the intersection of these genes with Visium genes (31,053), corresponding to 1408 genes.

Mapping Allen atlas data. Applicants used 58,022 10Xv3 snRNA-seq data for 83, 38 and 43 spatial spots, respectively, in the anterior, mid and posterior ROIs. Among 26,944 transcripts in the snRNA-seq data, 1,408 marker genes were selected. Applicants mapped using the intersection between these genes with Allen atlas genes measured coronally (overall, 4,345 genes); the intersection corresponds to 750 genes. The algorithm converged after 150 epochs.

Data Collection—snRNA-Seq Data and Histological Images

Mouse experiments. Mice were group housed with a 12-hour light-dark schedule and allowed to acclimate to their housing environment for two weeks post arrival. All procedures involving animals at MIT were conducted in accordance with the US National Institutes of Health Guide for the Care and Use of Laboratory Animals under protocol number 1115-111-18 and approved by the Massachusetts Institute of Technology Committee on Animal Care. All procedures involving animals at the Broad Institute were conducted in accordance with the US National Institutes of Health Guide for the Care and Use of Laboratory Animals under protocol number 0120-09-16.

Brain preparation prior to anatomical dissection and snRNA-seq. At 60 days of age, C57BL/6J mice (50% males, 50% females) were anesthetized by administration of isoflurane in a gas chamber flowing 3% isoflurane for 1 minute. Anesthesia was confirmed by checking for a negative tail pinch response. Animals were moved to a dissection tray and anesthesia was prolonged via a nose cone flowing 3% isoflurane for the duration of the procedure. Transcardial perfusions were performed with ice cold pH 7.4 HEPES buffer containing 110 mM NaCl, 10 mM HEPES, 25 mM glucose, 75 mM sucrose, 7.5 mM MgCl₂, and 2.5 mM KCl to remove blood from the brain and other organs sampled. The brain was removed immediately and frozen for 3 minutes in liquid nitrogen vapor and moved to −80° C. for long term storage. A detailed protocol is available at protocols.io (dx.doi.org/10.17504/protocols.io.bcbrism6).

Generation of MOp dissectates and snRNA-seq. Frozen mouse brains were securely mounted by the cerebellum onto cryostat chucks with OCT embedding compound such that the entire anterior half, including the primary motor cortex (MOp), was left exposed and thermally unperturbed. Dissection of 3 consecutive 500 μm anterior-posterior (A-P) spans of the MOp was performed by hand in the cryostat using an ophthalmic microscalpel (Feather safety Razor #P-715) precooled to −20° C. and donning 4× surgical loupes. Each 500 μm step was accomplished by advancing the cryostat (Leica CM3050S) 100 μm 5 times in trimming mode and cutting out each dissectate 100 μm at a time. This stepwise approach serves to ameliorate disruption of the brain tissue surface that occurs with large steps. Each excised tissue dissectate pool was placed into a pre-cooled 0.25 ml PCR tube using pre-cooled forceps and stored at −80° C. In order to assess dissection accuracy, 10 μm coronal registration sections were taken at each of the 500 μm A-P dissection junctions and imaged following Nissl staining. Nuclei were extracted from the frozen tissue dissectates using gentle, detergent-based dissociation, according to a protocol (dx.doi.org/10.17504/protocols.io.bbseinbe) adapted from one generously provided by the McCarroll lab, and loaded into the 10x Chromium V3 system (10× Genomics). Reverse transcription and library generation were performed according to the manufacturer's protocol.

snRNA-seq data were analyzed using the scanpy package. snRNA-seq data were pre-processed via the following steps: Applicants removed cells with high mitochondrial gene content; Applicants normalized the data to correct for library-size; Applicants applied the function f(x)=log (1+x) to the normalized counts. The resulting snRNA-seq data are ready to be mapped with Tangram. To compute marker genes of snRNA-seq data, Applicants applied a computational pipeline described in the tutorial of the scanpy package available at scanpy-tutorials.readthedocs.io/en/latest/pbmc3k.html.

Applicants used normalized quantities to visualize gene expression via mRNA counts (FIGS. 6b, 6c, 6f, 7c, 7e, 7f, 8c, 8d, 8e, 9e, 9g, 9h, 10c and 10d), gene expression via fluorescence (FIGS. 7c, 7f, 8c, 9h, and 10c) chromatin accessibility via ATAC peak counts (FIGS. 6f and 6c), and transcription factor activity via z-scores (FIG. 10d). Normalization is performed by rescaling the colorbar in each image, so that the minimum (respectively, maximum) value of the image correspond to the color with minimum (respectively, maximum) value in the colorbar. This is the default behavior of the plotting functions of the Python library matplotlib (matplotlib.org) which Applicants used throughout the manuscript.

### Data Collection—Visium

Mice. All mouse work was performed in accordance with the Institutional Animal Care and Use Committees (IACUC) and relevant guidelines at the Broad Institute, with protocol IACUC 0147-02-17.

Tissue processing. Fresh-frozen wild type C57BL/6 whole mouse brain was embedded in OCT (TissueTek Sakura) and cryo sectioned at 10 μm thickness at −20° C. Tissue sections were placed in 6.5 mm squared capture areas on pre-cold Visium Tissue Optimization slides (3000394, 10× Genomics) and Visium Spatial Gene Expression slides (2000233, 10× Genomics) and adhered by warming the backside of the slides and placed at −80° C. for up to 3 days.

Visium spatial gene expression library generation. The tissue optimization sample slide and spatial gene expression slide were processed according to the manufacturer's protocols (CG000238_VisiumSpatialTissueOptimizationUserGuide_Rev_A.pdf and CG000239_VisiumSpatialGeneExpression_UserGuide_Rev_A.pdf). Briefly, tissue sections were warmed to 37° C. for 1 minute and fixed for 30 min in ice-cold methanol, followed by 1 min isopropanol incubation at room temperature. Tissues were then hematoxylin and eosin (H&E) stained according to the protocol. Morphology brightfield images were taken with a Zeiss Axio microscope with the Metafer slide-scanning platform (Metasystems) with a 10× objective. For the tissue optimization slide fluorescent images, a TRITC filter and 10× resolution was used. Images were joined together with the VSlide software (Metasystems) and exported as tiff files. To optimize tissue permeabilization time, six different time points with 3-minute increments were tested on the tissue optimization sample slide. 12 minutes of permeabilization was used for the spatial gene expression slide. RNA released from the tissue was converted to cDNA by priming to the spatial barcoded primers on the glass via reverse transcription in the presence of template switching oligo, to generate full-length, spatially barcoded, UMI containing cDNA. Subsequently, following second strand synthesis, a denaturation step released the cDNA, followed by PCR amplification. Finally, sequencing-ready, indexed spatial gene expression libraries were constructed. Two of the libraries were pooled together and sequenced on a NextSeq 500/550 High output kit at 1.8 pM concentration. The sequencing settings were: read 1, 28 cycles; read 2, 91 cycles; index 1, 8 cycles.

MOp Visium raw read processing. Raw FASTQ files generated by Illumina's BCL2FASTQ conversion and the histology H&E images were provided as input to the SpaceRanger software (10x Genomics) version 1.1.0, available at support. 10×genomics. com/spatial-gene-expression/software/downloads/latest. Sequencing reads were mapped to the mm10 reference mouse genome using STARv2.5 mapping as part of SpaceRanger suite. Spatial barcodes were assigned by SpaceRanger to the barcoded spatial spots and aligned with the tissue image with the aid of the fiducial frames. Barcodes/UMI and genes were counted for the individual spots to generate an output gene expression per-spot matrix used as input for downstream data analysis.

MOp MERFISH data preprocessing. Applicants obtained MERFISH data from the Zhuang lab. Applicants preprocessed the data to remove sub-cortical cells. To identify sub-cortical cells, Applicants identify cells overly expressing Nxph4 (a marker gene of L6b region) and fit those cells with a square root polynomial. All cells below the fit were considered sub-cortical and removed.

### Image Datasets for Registration Pipeline

To locate ROIs, Applicants used images of Nissl-stained coronal mouse brain slices collected in the Macosko lab. To train and test the models presented in FIG. 11-13, Applicants used the following public image datasets:
(dataset avg): 1,320 images/segmentation masks of coronal slices from the average template of the Allen adult mouse brain atlas at resolution 10 μm (available at download.alleninstitute.org/informatics-archive/current-release/mouse_ccf/average_template/slice_images/).
(dataset ara): 1,320 images/segmentation masks of coronal slices from the Niss1 template of the Allen adult mouse brain atlas at resolution 10 μm (available at download.alleninstitute.org/informatics-archive/current-release/mouse_ccf/ara_niss1/).
(dataset p56c): 132 images/segmentation masks of coronal slices from the Allen P56 coronal reference atlas (available at mouse.brain-map.org/experiment/thumbnails/100048576?image_type=atlas).
(dataset p56d): 504 images of coronal slices from the Allen Development Atlas P56(available at help.brain-map.org/display/atlasviewer/Allen+Developing+Mouse+Brain+Atlas).
(dataset brainmaps): 111 images of coronal slices from Nissl-stained BrainMaps atlas (ID: 43) (available at brainmaps.org/index.php?action=viewslides&datid=43), and 87 images of coronal slices from Niss1-stained BrainMaps atlas (ID: 38) (available at brainmaps. org/index.php?action=viewslides&datid=38).
(dataset ish): 30 images of coronal slices from the Allen ISH Data (available at . mouse.brain-map.org/search/index).

Siamese network model for depth calling. Applicants used datasets avg, ara, p56c and p56d for training. Training images were resized to 224×224 and casted to type float32. Pixel values were rescaled between zero and one, prior to training. All images were augmented using imgaug (github.com/imgaug) library. As training labels Applicants used numerical coordinates, indicating the spatial coronal depth (i.e., posterior) of each mouse brain image on a scale of 10 μm. For the avg and ara datasets, labels were readily available from their tensor coordinates. Labels for the p56c and p56d datasets were also readily obtained using the AllenSDK API (allensdk.readthedocs.io/en/latest/). Dataset brainmaps and ish were manually annotated and used as test sets.

In designing the Siamese network model, Applicants used a DenseNet169 encoder pretrained on the ImageNet dataset and open-sourced through Keras Applications. Applicants fine-tuned the encoder by training the last convolutional layer. Applicants added two fully connected layers on top of the encoder in order to map the extracted features to the 512-dimensional latent space. A last fully-connected layer was used to map the latent space to the model output as represented in FIG. 11. All fully connected layers were trained.

A training sample consisted of two random images from the annotated datasets. The difference in spatial depth coordinates between the two images, denotes by \hat{d}ₗ, was used as the label. For example, if the first image were at posterior (depth) 500 μm and the second at a posterior 700 μm the corresponding label would be \hat{d}ₗ=200. Applicants used as penalty the mean squared error between the spatial depth difference predicted by the network dᵢ , and the labels \hat{d}ₗ:

$$MSE ( d , \hat{d} ) = \frac{1}{N} {\sum }_{i = 1}^{N} {( {d}_{i} - \hat{{d}_{i}} )}^{2} ,$$

where N indicates the number of training samples. Applicants trained the model for 50 epochs using 18,000 image pairs per epoch, partitioned to batches of 16 images.

Semantic segmentation model for anatomical region calling. Applicants used datasets avg, ara and p56c as training sets, since masks were available. Training images were resized to 512×512 and casted to type float32. Pixel values were rescaled between zero and one. As labels, Applicants used superimposable segmentation masks with the same dimension as the training images. Each mask was one-hot encoded into a 5-channel tensor to annotate each pixel into five different classes (FIG. 12): background (black), cortex (green), cerebellum (yellow), other grey matter (grey), and white matter (brown). Applicants used colors consistent with the Allen ontology to facilitate registration. For avg and ara datasets, Applicants used masks from the Allen CCFv3 ontology 2017 (available at download. alleninstitute. org/informatics-archive/current-release/mouse_ccf/annotation/ccf_2017/annotation_10.nrrd). For the p56c dataset, Applicants downloaded the SVG masks from the Allen Institute website, and rendered them into images using the library released in this study, which builds on Cairo SVG (cairosvg.org). Both images and masks were augmented using the same pipeline adopted for the Siamese model. In transforming the masks, Applicants ensured that the one-hot structure was preserved in the masks after augmentation.

Applicants used a semantic segmentation model from the Tensorflow Keras version of the segmentation_models library (github.com/qubvel/segmentation_models). Specifically, Applicants chose a U-NET architecture with a ResNet50 backbone²⁶. All weights have been randomly initialized following the He scheme, with the exception of the ResNet50 encoder which was pre-trained on ImageNet. The model was trained to optimize the superposition of the cross entropy and Jaccard index (i.e. intersection-over-union). The loss function is defined as:

$$L ( g , p ) = - g \cdot log ( p ) - \frac{p ⋂ g}{p ⋃ g} .$$

Where ga is the ground truth image and p is the corresponding model prediction. The model last unit employs a softmax activation function, thus outputting the probability of each pixel to be in each of the five classes. By applying an argmax function, Applicants assign each pixel to its most probable class. Finally, Applicants relied on test-time augmentation to increase model performances: each test image was augmented twelve times, and final predictions were de-augmented and averaged.

### Data Availability.

smFISH data, Visium VISp data, MERFISH MOp data and Smart-Seq2 VISp snRNA-seq data are available at github.com/spacetx-spacejam/data. MERFISH MOp data are available at download.brainimagelibrary.org:8811/02/26/02265ddb0dae51de/SHARE-seq dataset is available at GSE (associated manuscript in review). The STARmap dataset is publicly available at². All other data are available at: console.cloud.google.com/storage/browser/tommaso-brain-data.

### Code Availability.

Code is available on GitHub at github.com/orgs/broadinstitute/teams/tommaso_team/; see also, github.com/broadinstitute/Tangram.

### REFERENCES

1. Chen, K. H., Boettiger, A. N., Moffitt, J. R., Wang, S. & Zhuang, X. Spatially resolved, highly multiplexed RNA profiling in single cells. Science 348, (2015).
2. Wang, X. et al. Three-dimensional intact-tissue sequencing of single-cell transcriptional states. Science 361, eaat5691 (2018).
3. Codeluppi, S. et al. Spatial organization of the somatosensory cortex revealed by cyclic smFISH. biorxiv.org/lookup/doi/10.1101/276097 (2018) doi:10.1101/276097.
4. Stahl, P. L. et al. Visualization and analysis of gene expression in tissue sections by spatial transcriptomics. Science 353, 78-82 (2016).
5. Ma, S. et al. Chromatin potential identified by shared single cell profiling of RNA and chromatin. bioRxiv 2020.06.17.156943 (2020) doi:10.1101/2020.06.17.156943.
6. Regev, A. et al. The Human Cell Atlas. eLife 6, e27041 (2017).
7. Regev, A. et al. The Human Cell Atlas White Paper. ArXiv181005192 Q-Bio (2018).
8. Rozenblatt-Rosen, O., Stubbington, M. J. T., Regev, A. & Teichmann, S. A. The Human Cell Atlas: from vision to reality. Nat. News 550, 451 (2017).
9. Rood, J. E. et al. Toward a Common Coordinate Framework for the Human Body. Cell 179, 1455-1467 (2019).
10. Lahnemann, D. et al. Eleven grand challenges in single-cell data science. Genome Biol. 21, 31 (2020).
11. Macosko, E. Z. et al. Highly Parallel Genome-wide Expression Profiling of Individual Cells Using Nanoliter Droplets. Cell 161, 1202-1214 (2015).
12. Kotliar, D. et al. Identifying Gene Expression Programs of Cell-type Identity and Cellular Activity with Single-Cell RNA-Seq. bioRxiv (2018) doi:10.1101/310599.
13. Smillie, C. S. et al. Intra- and Inter-cellular Rewiring of the Human Colon during Ulcerative Colitis. Cell 178, 714-730.e22 (2019).
14. Haghverdi, L., Büttner, M., Wolf, F. A., Buettner, F. & Theis, F. J. Diffusion pseudotime robustly reconstructs lineage branching. Nat. Methods 13, 845-848 (2016).
15. La Manno, G. et al. RNA velocity of single cells. Nature 560, 494-498 (2018).
16. Ke, R. et al. In situ sequencing for RNA analysis in preserved tissue and cells. Nat. Methods 10, 857-860 (2013).
17. Codeluppi, S. et al. Spatial organization of the somatosensory cortex revealed by osmFISH. Nat. Methods 15, 932-935 (2018).
18. Alon, S. et al. Expansion Sequencing: Spatially Precise In Situ Transcriptomics in Intact Biological Systems. biorxiv.org/lookup/doi/10.1101/2020.05.13.094268 (2020) doi:10.1101/2020.05.13.094268.
19. Eng, C.-H. L. et al. Transcriptome-scale super-resolved imaging in tissues by RNA seqFISH+. Nature (2019) doi:10.1038/s41586-019-1049-y.
20. Rodrigues, S. G. et al. Slide-seq: A scalable technology for measuring genome-wide expression at high spatial resolution. Science 363, 1463-1467 (2019).
21. Vickovic, S. et al. High-definition spatial transcriptomics for in situ tissue profiling. Nat. Methods 16, 987-990 (2019).
22. Satija, R., Farrell, J. A., Gennert, D., Schier, A. F. & Regev, A. Spatial reconstruction of single-cell gene expression data. Nat. Biotechnol. 33, 495-502 (2015).
23. Achim, K. et al. High-throughput spatial mapping of single-cell RNA-seq data to tissue of origin. Nat. Biotechnol. 33, 503-509 (2015).
24. Stuart, T. et al. Comprehensive integration of single cell data. biorxiv.org/lookup/doi/10.1101/460147 (2018) doi:10.1101/460147.
25. Nitzan, M., Karaiskos, N., Friedman, N. & Rajewsky, N. Gene expression cartography. Nature 576, 132-137 (2019).
26. Andersson, A. et al. Spatial mapping of cell types by integration of transcriptomics data. biorxiv.org/lookup/doi/10.1101/2019.12.13.874495 (2019) doi:10.1101/2019.12.13.874495.
27. Maaskola, J. et al. Charting Tissue Expression Anatomy by Spatial Transcriptome Decomposition. biorxiv.org/lookup/doi/10.1101/362624 (2018) doi:10.1101/362624.
28. Wang, Q. et al. The Allen Mouse Brain Common Coordinate Framework: A 3D Reference Atlas. Cell 181, 936-953.e20 (2020).
29. Tustison, N. J. et al. Large-scale evaluation of ANTs and FreeSurfer cortical thickness measurements. Neurolmage 99, 166-179 (2014).
30. Balakrishnan, G., Zhao, A., Sabuncu, M. R., Guttag, J. & Dalca, A. V. VoxelMorph: A Learning Framework for Deformable Medical Image Registration. IEEE Trans. Med. Imaging 38, 1788-1800 (2019).
31. Chen, Y. et al. An active texture-based digital atlas enables automated mapping of structures and markers across brains. Nat. Methods 16, 341 (2019).
32. Ding, J. et al. Systematic comparison of single-cell and single-nucleus RNA-sequencing methods. Nat. Biotechnol. 38, 737-746 (2020).
33. Tasic, B. et al. Shared and distinct transcriptomic cell types across neocortical areas. Nature 563, 72-78 (2018).
34. Yao, Z. et al. An integrated transcriptomic and epigenomic atlas of mouse primary motor cortex cell types. biorxiv.org/lookup/doi/10.1101/2020.02.29.970558 (2020) doi:10.1101/2020.02.29.970558.
35. Picelli, S. et al. Full-length RNA-seq from single cells using Smart-seq2. Nat. Protoc. 9, 171-181 (2014).
36. V1_Adult_Mouse_Brain-Datasets-Spatial Gene Expression-Official 10× Genomics Support. support.10xgenomics.com/spatial-gene-expression/datasets/1.1.0/V1_Adult_Mouse_Brain?
37. Buenrostro, J. D. et al. Single-cell chromatin accessibility reveals principles of regulatory variation. Nature 523, 486-490 (2015).
38. Kozareva, V. et al. A transcriptomic atlas of the mouse cerebellum reveals regional specializations and novel cell types. bioRxiv 2020.03.04.976407 (2020) doi:10.1101/2020.03.04.976407.
39. Stickels, R. R. et al. Sensitive spatial genome wide expression profiling at cellular resolution. bioRxiv 2020.03.12.989806 (2020) doi:10.1101/2020.03.12.989806.
40. Stoeckius, M. et al. Simultaneous epitope and transcriptome measurement in single cells. Nat. Methods 14, 865-868 (2017).
41. Cadwell, C. R. et al. Electrophysiological, transcriptomic and morphologic profiling of single neurons using Patch-seq. Nat. Biotechnol. 34, 199-203 (2016).
42. Welch, J. D., Hartemink, A. J. & Prins, J. F. MATCHER: manifold alignment reveals correspondence between single cell transcriptome and epigenome dynamics. Genome Biol. 18, 138 (2017).
43. Nowotschin, S. et al. The emergent landscape of the mouse gut endoderm at single-cell resolution. Nature 569, 361-367 (2019).
44. Thornton, C. A. et al. Spatially-mapped single-cell chromatin accessibility. bioRxiv 815720 (2019) doi:10.1101/815720.
45. Wolf, F. A., Angerer, P. & Theis, F. J. SCANPY: large-scale single-cell gene expression data analysis. Genome Biol. 19, 15 (2018).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A method of aligning single cell data with spatial data to generate spatial maps of cell types and gene expression at single cell resolution comprising:
by one or more computing devices:
receiving single cell data obtained from a specimen from a specific anatomical region or tissue type;
receiving spatial data obtained from the same specimen or the same anatomical region or tissue type from a different specimen,
wherein the single cell data and spatial data comprises at least one shared measured features;
aligning the single cell data to the spatial data based on one or more of the shared features using unsupervised deep learning nonlinear optimization; and
generating a spatial map wherein the single cell data constitutes the new spatial data.

2. The method of claim 1, wherein the unsupervised deep learning non-linear optimization uses one or more similarity functions, preferably, wherein the one or more similarity functions comprise Kullback-Leibler (KL) divergence and cosine similarity.

3. (canceled)

4. The method of claim 1, further comprising validating the spatial map by predicting the expression of one or more holdout genes; and/or
further comprising applying a first learned spatial map to a second spatial map to increase the resolution of the second map; and/or
further comprising relating the spatial map to histological and anatomical data for the same specimen or the same anatomical region or tissue type from a different specimen; and/or
further comprising registering the spatial maps on an anatomically annotated common coordinate framework, preferably, wherein registering comprises using a Siamese neural network and a semantic segmentation algorithm.

5-8. (canceled)

9. The method of claim 1, wherein the shared features comprise gene expression, accessible chromatin, an epigenetic mark, and/or a combination thereof; and/or
wherein the single cell data and spatial data comprises at least 10 shared features; and/or
wherein the single cell data comprises the expression of greater than 10,000 genes and the spatial data comprises spatial expression of less than 100 genes; and/or
wherein the single cell data comprises single cell RNA-seq data (scRNA-seq) or single nucleus RNA-seq data (snRNA-seq); and/or
wherein the single cell data comprises single cell ChIP; and/or
wherein the single cell data comprises single cell ATAC-seq; and/or
wherein the single cell data comprises single cell proteomics; and/or
wherein the spatial data is coarse grained; and/or
wherein the spatial data comprises in situ Hybridization (ISH), Single-molecule Fluorescence in situ Hybridization (smFISH), Spatial Transcriptomics (Visium), STARmap and/or MERFISH; and/or
wherein the single cell data is multi-modal single cell data, preferably, wherein the multi-modal data is single cell RNA-seq and chromatin accessibility data (SHARE-seq); or wherein the multi-modal data is single cell RNA-seq and proteomics data (CITE-seq); or wherein the multi-modal data is single cell RNA-seq and patch-clamping electrophysiological recording and morphological analysis of single neurons (Patch-seq).

10-21. (canceled)

22. The method of claim 1, comprising receiving an input of one or more types of genes.

23. The method of claim 1, comprising generating a single cell matrix and a mapping matrix, preferably,
wherein the method comprises:
determining a quantity of cells in the single cell data;
determining a quantity of cells in the spatial data; and
applying a mapping filter if the quantity of cells in the single cell data is greater than the quantity of cells in the spatial data, more preferably,
wherein applying the mapping filter comprises:
determining a probability of finding each cell of the single cell data in a voxel of the spatial data;
assigning a filter value to each cell of the single cell data based on the determined probability;
generating a filter vector;
applying a sigmoid function to the filter vector; and
filtering the single cell matrix and the mapping matrix.

24-25. (vanceled)

26. A system to align single cell data with spatial data to generate spatial maps of cell types at single cell resolution comprising:
a storage device; and
a processor communicatively coupled to the storage device, wherein the processor executes application code instructions that are stored in the storage device to cause the system to:
receive single cell data obtained from a specimen from a specific anatomical region or tissue type;
receive spatial data obtained from the same specimen or the same anatomical region or tissue type from a different specimen,
wherein the single cell data and spatial data comprises at least one shared feature;
align the single cell data to the spatial data based on one or more of the shared features using unsupervised deep learning non linear optimization; and
generate a spatial map wherein the single cell data constitutes the new spatial data.

27. The system of claim 26, wherein the unsupervised deep learning non-linear optimization uses one or more similarity functions, preferably, wherein the one or more similarity functions comprise Kullback-Leibler (KL) divergence and cosine similarity.

28. (canceled)

29. The system of claim 26, further comprising application code instructions to validate the spatial map by predicting the expression of one or more holdout genes; and/or
further comprising application code instructions to apply a first learned spatial map to a second spatial map to increase the resolution of the second map; and/or
further comprising application code instructions to relate the spatial map to histological and anatomical data for the same specimen or the same anatomical region or tissue type from a different specimen; and/or
further comprising application code instructions to register the spatial maps on an anatomically annotated common coordinate framework, preferably, wherein registering comprises application code instructions to use a Siamese neural network and a semantic segmentation algorithm.

30-33. (canceled)

34. The system of claim 26, wherein the shared features comprise gene expression, accessible chromatin, an epigenetic mark, and/or a combination thereof; and/or
wherein the single cell data and spatial data comprises at least 10 shared features; and/or
wherein the single cell data comprises the expression of greater than 10,000 genes and the spatial data comprises spatial expression of less than 100 genes; and/or
wherein the single cell data comprises single cell RNA-seq data (scRNA-seq) or single nucleus RNA-seq data (snRNA-seq); and/or
wherein the single cell data comprises single cell ChIP; and/or
wherein the single cell data comprises single cell ATAC-seq; and/or
wherein the single cell data comprises single cell proteomics; and/or
wherein the spatial data is coarse grained; and/or
wherein the spatial data comprises in situ Hybridization (ISH), Single-molecule Fluorescence in situ Hybridization (smFISH), Spatial Transcriptomics (Visium), STARmap and/or MERFISH; and/or
wherein the single cell data is multi-modal single cell data, preferably, wherein the multi-modal data is single cell RNA-seq and chromatin accessibility data (SHARE-seq); or wherein the multi-modal data is CITE-seq data; or wherein the multi-modal data is single cell RNA-seq and patch-clamping electrophysiological recording and morphological analysis of single neurons (Patch-seq).

35-46. (Canceled)

47. The system of claim 26, comprising application code instructions to receive an input of one or more types of genes.

48. The system of claim 26, comprising application code instructions to generate a single cell matrix and a mapping matrix, preferably,
wherein the system comprises application code instructions to:
determine a quantity of cells in the single cell data;
determine a quantity of cells in the spatial data; and
apply a mapping filter if the quantity of cells in the single cell data is greater than the quantity of cells in the spatial data, more preferably,
wherein applying the mapping filter comprises application code instructions to:
determine a probability of finding each cell of the single cell data in a voxel of the spatial data;
assign a filter value to each cell of the single cell data based on the determined probability;
generate a filter vector;
apply a sigmoid function to the filter vector; and
filter the single cell matrix and the mapping matrix.

49-50. (canceled)

51. A computer program product to align single cell data with spatial data to generate spatial maps of cell types at single cell resolution, comprising:
a non-transitory computer-readable medium having computer-readable program instructions embodied thereon that, when executed by a computer, cause the computer to:
receive single cell data obtained from a specimen from a specific anatomical region or tissue type;
receive spatial data obtained from the same specimen or the same anatomical region or tissue type from a different specimen,
wherein the single cell data and spatial data comprises at least one shared feature;
align the single cell data to the spatial data based on one or more of the shared features using unsupervised deep learning non linear optimization; and
generate a spatial map wherein the single cell data constitutes the new spatial data.

52. The computer program product of claim 51, wherein the unsupervised deep learning non-linear optimization uses one or more similarity functions, preferably, wherein the one or more similarity functions comprise Kullback-Leibler (KL) divergence and cosine similarity.

53. (canceled)

54. The computer program product of claim 51, further comprising computer-readable program instructions to validate the spatial map by predicting the expression of one or more holdout genes; and/or
further comprising computer-readable program instructions to apply a first learned spatial map to a second spatial map to increase the resolution of the second map; and/or
further comprising computer-readable program instructions to relate the spatial map to histological and anatomical data for the same specimen or the same anatomical region or tissue type from a different specimen; and/or
further comprising computer-readable program instructions to register the spatial maps on an anatomically annotated common coordinate framework, preferably, wherein registering comprises computer-readable program instructions to use a Siamese neural network and a semantic segmentation algorithm.

55-58. (canceled)

59. The computer program product of claim 51, wherein the shared features comprise gene expression, accessible chromatin, an epigenetic mark, and/or a combination thereof; and/or
wherein the single cell data and spatial data comprises at least 10 shared features; and/or
wherein the single cell data comprises the expression of greater than 10,000 genes and the spatial data comprises spatial expression of less than 100 genes; and/or
wherein the single cell data comprises single cell RNA-seq data (scRNA-seq) or single nucleus RNA-seq data (snRNA-seq); and/or
wherein the single cell data comprises single cell ChIP; and/or
wherein the single cell data comprises single cell ATAC-seq; and/or
wherein the single cell data comprises single cell proteomics; and/or
wherein the spatial data is coarse grained; and/or
wherein the spatial data comprises in situ Hybridization (ISH), Single-molecule Fluorescence in situ Hybridization (smFISH), Spatial Transcriptomics (Visium), STARmap and/or MERFISH; and/or
wherein the single cell data is multi-modal single cell data, preferably, wherein the multi-modal data is single cell RNA-seq and chromatin accessibility data (SHARE-seq); or wherein the multi-modal data is CITE-seq data; or wherein the multi-modal data is single cell RNA-seq and patch-clamping electrophysiological recording and morphological analysis of single neurons (Patch-seq).

60-71. (canceled)

72. The computer program product of claim 51, comprising computer-readable program instructions to receive an input of one or more types of genes.

73. The computer program product of claim 51, comprising computer-readable program instructions to generate a single cell matrix and a mapping matrix, preferably,
wherein the computer program product comprises computer-readable program instructions to:
determine a quantity of cells in the single cell data;
determine a quantity of cells in the spatial data; and
apply a mapping filter if the quantity of cells in the single cell data is greater than the quantity of cells in the spatial data, more preferably,
wherein applying the mapping filter comprises computer-readable program instructions to:
determine a probability of finding each cell of the single cell data in a voxel of the spatial data;
assign a filter value to each cell of the single cell data based on the determined probability;
generate a filter vector;
apply a sigmoid function to the filter vector; and
filter the single cell matrix and the mapping matrix.

74-75. (canceled)
